(19) United States
(12) Patent Application Publication
(10) Pub. No.: US 2016/0278713 A1
(43) Pub. Date: Sep. 29, 2016

(54) COMPACT LOW-POWER RECORDING ARCHITECTURE FOR MULTICHANNEL ACQUISITION OF BIOLOGICAL SIGNALS AND METHOD FOR COMPRESSING SAID BIOLOGICAL SIGNAL DATA

(71) Applicant: ECOLE POLYTECHNIQUE FEDERALE DE LAUSANNE (EPFL), Lausanne (CH)

(72) Inventors: Mahsa SHOARAN, St-Sulpice VD (CH); Mahdad HOSSEINI KAMAL, Lausanne (CH); Alexandre SCHMID, Sion (CH)

(21) Appl. No.: 14/668,313

(22) Filed: Mar. 25, 2015

(51) Int. Cl. A61B 5/00; A61B 5/0478

(52) U.S. Cl. CPC A61B 5/7232; A61B 5/0006; A61B 5/0478; A61B 5/725

(57) ABSTRACT

The present invention relates to a method for compressing signal data of a plurality of biological signals captured by a plurality of implanted measurement devices. The present invention further relates to an implantable sensor configured to carry out said method of compression. The present invention also concerns a method for recovering the plurality of biological signals from the compressed data as well as a system including a plurality of implantable sensors, a transmitter, a receiver and a processor configured to recover the plurality of biological signals from the compressed data.

### FIELD OF THE INVENTION

The present invention relates to a method for compressing signal data of a plurality of biological signals captured by a plurality of implanted measurement devices. The present invention further relates to an implantable sensor configured to carry out said method of compression. The present invention also concerns a method for recovering the plurality of biological signals from the compressed data as well as a system including a plurality of implantable sensors, a transmitter, a receiver and a processor configured to recover the plurality of biological signals from the compressed data.

More particularly, the present invention introduces an area- and power-efficient approach for compressive recording of, for example, cortical signals used in an implantable system prior to transmission. Recent research on compressive sensing has shown promising results for sub-Nyquist sampling of sparse biological signals. Still, any large-scale implementation of this technique faces critical issues caused by the increased hardware intensity. The cost of implementing compressive sensing in a multichannel system in terms of area usage can be significantly higher than a conventional data acquisition system without compression. To tackle this issue, the present invention concerns a new multichannel compressive sensing scheme which exploits the spatial sparsity of the signals recorded from the electrodes of the sensor array. The analysis shows that using this method, the power efficiency is preserved to a great extent while the area overhead is significantly reduced resulting in an improved power-area product. The proposed circuit architecture is implemented in a UMC 0.18 μm CMOS technology. Extensive performance analysis and design optimization has been done resulting in a low-noise, compact and power-efficient implementation. The results of simulations and subsequent reconstructions show the possibility of recovering fourfold compressed intracranial EEG signals with an SNR as high as 21.8 dB, while consuming 10.5 μW of power within an effective area of 250 μm×250 μm per channel.

### BACKGROUND

### I. Introduction

Wireless monitoring of brain activity through implantable devices is a promising technology enabling advanced and cost-effective diagnosis and treatment of brain disorders such as stroke, Parkinson's disease, depression and epilepsy [1]-[3]. Recording from multiple sites, however, introduces a major technological bottleneck as the large bandwidth requirement for data telemetry which is not easily achievable by state-of-the-art wireless technology. The increased power consumption of transmission for large recording arrays can cause major safety and biocompatibility concerns regarding the applicability of such devices. Thus, some type of data reduction prior to telemetry is needed to meet the requirements of an implantable device.

Compressive sensing (CS) [4], [5] is an emerging compression method with interesting advantages over traditional methods thanks to its low encoder complexity and universality with respect to the signal model. A compressive sensing system samples a high-dimensional signal into a smaller number of linear measurements than dictated by the Nyquist sampling theorem. CS has been recently studied in the context of biological signals (e.g., ECG [6], [7], EEG [8] and iEEG [9]) to tackle the data rate issue. When compared to thresholding and activity-dependent recording, CS has the advantage of preserving the temporal information and morphology of the signal for the entire recording period. It is also possible to apply CS along with other methods (such as interpacket redundancy removal, Huffman coding [6] or dynamic power management of the front-end LNA (low-noise amplifier)) in order to further relax the stringent energy and bandwidth requirements of implantable system.

While the majority of research presented in literature focus on power minimization of the implantable system, there is also a stringent need to minimize the circuit area in order to include the highest number of recording units into the available die area. Large-scale recording of cortical activity is particularly important in the case of diseases like epilepsy which spread over wide regions of cortical area. Conventional subdural electrodes are used in such applications which consist of several conductive discs mounted in thin plastic and designed to lie on the surface of the cortex. However, the state-of-the-art research targeting such applications progresses toward minimally invasive flexible and dense recording arrays with high-resolution recording capability of intracranial EEG (iEEG) signals [10]-[12]. The high resolution (i.e., small spacing of recording sites) provides the capability of capturing higher frequency activity than traditionally recordable by large widely-spaced electrodes, giving a profound insight into the fundamental mechanisms underlying such abnormalities. Electrocorticographic signals recorded from human cortex can be used as an alternative to invasive spike recordings through penetrating electrodes, in order to control prosthetic limbs in BMIs as shown in [13].

The common microelectronic approach to CS ([8], [14], [15]) consists of on-the-fly compression of consecutive samples of each recording unit over time, either in analog [FIG. 1(a)] or digital [FIG. 1(b)] domain. Even though this approach results in a significant energy efficiency, its large area usage disqualifies the concept for a multichannel recording interface which should include the circuits supporting many channels in a limited die area.

The present invention permits to overcome this issue by providing a new multichannel measurement scheme [see, for example, FIG. 1(c)] along with an appropriate recovery scheme, encoding the whole array into a single compressed data stream. In the proposed approach, the compression is carried out in the analog domain, and in a multichannel fashion. This technique advantageously circumvents the need to place one ADC per channel and results in a significant area saving. Based on this approach, a wireless monitoring system consisting of several recording/compressing units is proposed (FIG. 2). Taking benefit of the area-efficient implementation of CS, the number of recording units implantable, for example, on the cortex which satisfy the energy constraints of the system is scaled up by a factor equal to the compression ratio.

### SUMMARY OF THE INVENTION

The present invention relates to a method for compressing a plurality of biological signals provided by a plurality of measurement devices through a plurality of measuring channels, the method comprising the steps of forming a multichannel signal matrix XεR^{d×N} from the plurality of biological signals where d is a dimension of the biological signal in each channel and in a time-window and N is the number of channels, R meaning a set of real numbers; and obtaining a multichannel measurement vector Y by acquiring M=p/d measurements from each column of the multichannel signal matrix X, to acquire M measurements at each time-window from all channels, where p<<d×N.

The method further includes the step of applying a reshaping operator [symbol] :R^{d×N}→R^{d·N} to the multichannel signal matrix X to transpose said matrix to form a vector of concatenated columns of the multichannel signal matrix X.

The multichannel measurement vector Y is obtained by applying a measurement matrix Φ_{MC} to the vector of concatenated columns of the multichannel signal matrix X.

The measurement matrix φ_{MC} is represented in matrix form as follows:

$${Φ}_{MC} = [ \begin{matrix}{Φ}_{1} & 0 ⋯ & 0 \\ ⋮ & ⋱ & ⋮ \\ 0 & ⋯ & {Φ}_{d}\end{matrix} ] , {Φ}_{i} = [ \begin{matrix}{ϕ}_{i}^{1 , 1} & ⋯ & {ϕ}_{i}^{1 , N} \\ ⋮ & ⋱ & ⋮ \\ {ϕ}_{i}^{M , 1} & ⋯ & {ϕ}_{i}^{M , n}\end{matrix} ] ,$$

where φᵢεR^{M×N} and φᵢ^{k,l} is uniformly selected from {0,1}.

The multichannel measurement vector Y is determined as follows:

Y=φ_{MC} [symbol] (X).

The method further includes the step of recovering a multichannel signal \hat{X} from the multichannel measurement vector Y using an l_{1,2} mixed norm as follows:

$$\hat{X} = {Ψ}_{MC} {argmin}_{{α}_{MC} ∈ {R}^{d \cdot N}} \vert \vert {α}_{MC} {\vert \vert}_{1 , 2} s . t . Y = {Φ}_{MC} 𝒫 ( {Ψ}_{MC} {α}_{MC} )$$

where argmin is argument of the minimum, ∥•∥_{1,2} models group sparsity according to the l_{1,2} mixed norm, α_{MC} is a Gabor transform coefficients, [symbol] is the reshaping operator and ψ_{MC} is a sparsity matrix.

The method additionally or alternatively includes the step of recovering a multichannel neural signal \hat{X} from the multichannel measurement vector Y using an l₁ norm as follows:

$$\hat{X} = {argmin}_{X ∈ {R}^{d \times N}} \vert \vert {Ψ}_{MC}^{T} {X}_{vec} {\vert \vert}_{1} s . t . Y = {Φ}_{MC} 𝒫 ( X )$$

where X_{vec} is the vector form of X which includes the concatenation of its columns, and the l₁ norm of a vector ξ is defined as ∥ξ∥₁=Σᵢ|ξᵢ|.

The present invention also relates to an implantable sensor including circuitry configured to carry out the above method.

The present invention further relates to an implantable system including an implantable transmitter and at least one of said implantable sensors, said at least one implantable sensor being connected to the implantable transmitter to communicate the compressed signal to the implantable transmitter for transmission to an external receiver.

The present invention further concerns a system for compressing a plurality of biological signals and recovering biological signals, the system including the previously mentioned implantable system and a receiver for receiving a transmitted compressed signal.

The system further includes a processor configured to receive the compressed signal from the receiver and to process the compressed signal according to the above method to reconstruct the captured biological signals.

The present invention additionally concerns an implantable sensor including a plurality of electrodes for capturing a biological signal, a plurality of amplifiers, each amplifier being connected to a single electrode to amplify the signal captured by each electrode, a random summing circuit for receiving each of the amplified signals, for randomly selecting samples of the amplified captured signals and summing said samples to provide a multichannel measurement signal, and a single analog-to-digital converter for receiving and digitizing the multichannel measurement signal to provide a digital compressed signal.

In the implantable sensor, each amplifier is a low-noise amplifier including a band-pass transfer function.

The implantable sensor further includes a plurality of low-pass filters to limit a high cut-off frequency of the amplified captured signal, each low-pass filter being connected to a single amplifier to receive the amplified captured signal.

The implantable sensor further includes a plurality of additional amplifiers, each additional amplifier being connected to a single low-pass filter to amplify the signal provided by said low-pass filter.

The implantable sensor further includes a plurality of buffered sample-and-hold circuits, each buffered sample-and-hold circuit being connected to a single additional amplifier to receive the signal amplified by said additional amplifier.

The present invention additionally concerns an implantable system including an implantable transmitter and at least one of the above implantable sensors, the least one implantable sensor being connected to the implantable transmitter to communicate the compressed signal to the implantable transmitter for transmission to an external receiver.

The present invention additionally concerns a system for compressing a plurality of biological signals and recovering biological signals, the system including the previously mentioned implantable system and a receiver for receiving a transmitted compressed signal.

Said system further includes a processor configured to receive the compressed signal from the receiver and to process the compressed signal to reconstruct the captured biological signals.

### BRIEF DESCRIPTION OF THE DRAWINGS

The above object, features and other advantages of the present invention will be best understood from the following detailed description in conjunction with the accompanying drawings, in which:

FIGS. 1(a), 1(b), and 1(c) illustrate a block diagram of (a) the analog single-channel CS, (b) the digital single-channel CS, and (c) the proposed multichannel CS architectures according to the present invention where d=1024, N=16 and M=4;

FIG. 2 illustrates an exemplary system-level view of the proposed multichannel compressive sensing method used in an implantable neural recording interface [16], where the neural signals sensed at the electrode sites are amplified, randomly projected, summed up and digitized through a single on-chip ADC; an RF unit placed within a burr hole in the skull transmits the compressed and digitized data originating from several recording chips to an external receiver and powers the implanted system;

FIGS. 3(a), 3(b), and 3(c) illustrate the structure of Gabor coefficients of multichannel neural signals in a Gabor window where FIG. 3(a) shows original neural signals: the Gabor coefficients represent group structure along frequencies, FIG. 3 (b) shows the l₁ norm: the Gabor coefficients are scattered and recovery does not respect the group structure and FIG. 3(c) shows the l_{1,2} mixed norm: the joint recovery preserves the group structure of Gabor coefficients;

FIGS. 4(a), 4(b), 4(c), and 4(d) illustrate an exemplary circuit implementation of the proposed multichannel CS architecture where FIG. 4(a) shows individual channels, FIG. 4(b) shows a variable-gain summing stage, gain stage and 10-bit SAR ADC (successive-approximation-register analog-to-digital converter) with attenuated binary-weighted DAC (digital-to-analog converter), FIG. 4(c) shows a multi-output random sequence generator, and FIG. 4(d) illustrates a timing diagram of the system;

FIG. 5 consists of FIG. 5(a) that illustrates a compact low-noise differential amplifier and FIG. 5(b) that illustrates a half-circuit equivalent used for noise analysis;

FIG. 6 illustrates an architecture for realizing a high-density recording system where each block of this figure corresponds to one of the implantable chip-electrode combinations of FIG. 2 (excluding the electrodes);

FIG. 7 consists of FIGS. 7(a) and 7(b) that illustrate noise analysis models of the summing stage of FIG. 4 in (a) sampling phase and (b) integrating phase;

FIG. 8 shows a measured frequency response of a stand-alone channel;

FIG. 9 shows a measured input-referred noise of a stand-alone channel;

FIG. 10 consists of FIGS. 10(a) and 10(b). FIG. 10(a) shows a microphotograph of the chip and an individual channel's layout;

FIG. 10(b) shows a power breakdown of a 16-channel compressive sensing array;

FIG. 11 shows one channel of human intracranial EEG recording using strip and greed electrodes implanted on the left temporal lobe, where the recovery SNR is calculated by averaging over 100 blocks of signal in the low-voltage fast activity region;

FIGS. 12(a) and 12(b) shows a comparison of recovery performance using different reconstruction methods for a block of length 1024 and compression ratio of 4, where FIG. 12(a) shows l₁ recovery, SNR_{CH1}=21.3 dB and FIG. 12(b) shows l_{1,2} recovery, SNR_{CH1}=28.04 dB.

FIG. 13 shows a comparison of mixed norm and sparse recovery performance for different compression ratios where SNRs are averaged over 20 compression blocks;

FIG. 14 illustrates an exemplary neural signal acquisition model, where a 16-channel neural signal is compressed through a 4-measurement compressive acquisition system.

### DETAILED DESCRIPTION

### II. Theoretical Model

Compressive sensing exploits the known structures in the signals to lower the required sampling ratio below the Nyquist rate, while providing a signal recovery of acceptable quality [17]. The basic concepts of CS are provided in the following discussion, emphasizing the benefit of applying CS in a neural recorder array. A detailed explanation of CS can be found in [4], [5], [18].

### A. Compressive Sampling

Compressive signal acquisition prescribes sampling a signal xεR^{d} with a significantly smaller number of samples than the signal sampled at the Nyquist rate. The linear measurements yεR^{m}, m<<d are formed by computational projection of the signal onto the measurement matrix φεR^{m×d}. Compressive measurements over a defined time interval are obtained by

y=φx (1)

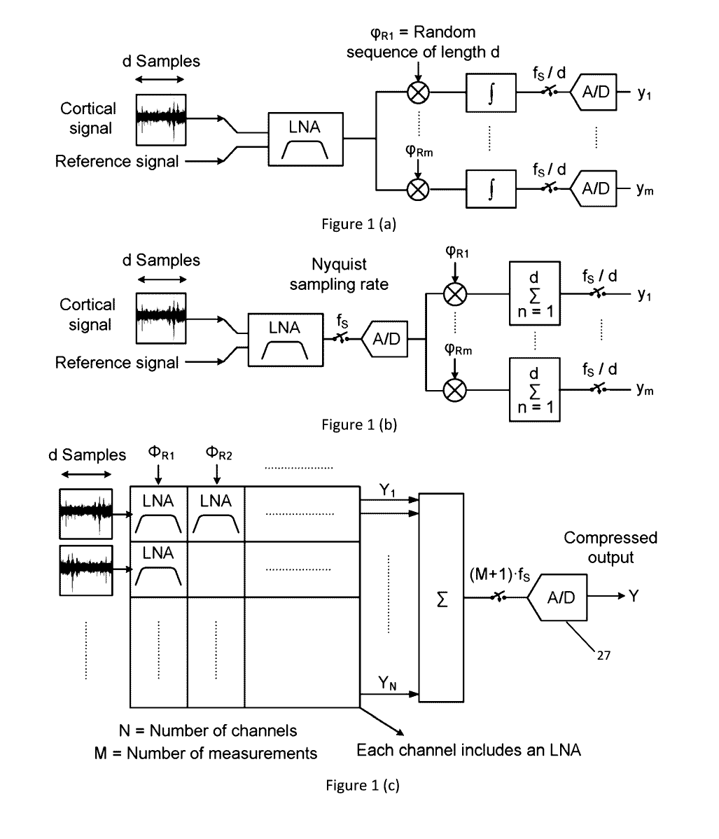

The corresponding block diagram is shown in FIGS. 1(a) and (b). The acquisition system is under-determined, i.e., the number of solutions for x is infinite. However, when the signal is known to be sparse in a basis ψεR^{d×d} in which x=ψα can be sparsely represented, the sparsest representation of x is the solution to the problem. A signal is said to be sparse in a basis if it can be represented by S non-zero coefficients, i.e., S<<d.

To ensure a successful recovery of x, the linear map φ should satisfy the Restricted Isometry Property (RIP) [19]. The measurement matrix satisfies the RIP for an S-sparse signal x (S non-zero coefficients) and a small restricted isometry constant δ_{S}<1 if

(1−δ_{S})∥x∥₂²≦∥φx∥₂²≦(1+δ_{S})∥x∥₂² (2)

∥x∥₂ represents the l₂ norm of x, which is defined as ∥x∥₂=(Σᵢ|xᵢ|²)^{1/2}. Sampling matrices with elements generated independently and according to a subgaussian distribution, such as the independent and identically distributed (i.i.d) Gaussian or the Bernoulli/Rademacher distributions (random 0 and 1), satisfy the RIP.

### B. Multichannel Acquisition Model of the Present Invention

In the case of multichannel neural recording, measurement limitations such as die area and power consumption suggest the use of a multichannel compression technique rather than acquiring each channel separately. Therefore, it is important to consider a measurement scheme which fulfills the physical constraints of the system. Let XεR^{d×N} represent the multichannel iEEG signal where d is the dimension of the signal in each channel and in a defined time-window called compression block and N is the number of channels. We define a reshaping operator [symbol] :R^{d×N}→R^{d·N} which transposes the input matrix and vectorizes the resulting matrix by concatenating its columns after each other. The linear compressive measurements are obtained by acquiring M=p/d measurements from columns of X, i.e., M measurement at each time-sample from all channels, where p<<d×N is the total number of measurements. Hence, the multichannel linear map can be represented in matrix form φ_{MC}εR^{(Md)×(dN)} as follows:

$${Φ}_{MC} = [ \begin{matrix}{Φ}_{1} & 0 ⋯ & 0 \\ ⋮ & ⋱ & ⋮ \\ 0 & ⋯ & {Φ}_{d}\end{matrix} ] , {Φ}_{i} = [ \begin{matrix}{ϕ}_{i}^{1 , 1} & ⋯ & {ϕ}_{i}^{1 , N} \\ ⋮ & ⋱ & ⋮ \\ {ϕ}_{i}^{M , 1} & ⋯ & {ϕ}_{i}^{M , n}\end{matrix} ] , \qquad ( 3 )$$

where φᵢεR^{M×N} and φᵢ^{k,l} is uniformly selected from {0,1} to approximate a measurement matrix similar to the Bernoulli matrix. The multichannel measurement vector YεR^{p} is defined as

Y=φ_{MC} [symbol] (X) (4)

### C. Sparse Recovery

The recovery of the multichannel iEEG signal from the compressive measurements explicitly employs a multichannel sparsity domain ψ_{MC}. The underlying neural signals in channels share similar structures. Hence, the transform domain of the multichannel signal is a block-diagonal matrix which presents the sparsity domain of channels along the diagonal and is defined as

ψ_{MC}=I_{N} [symbol] ψ (5)

ψεR^{k×d} is the sparsity domain of each channel, I_{N} is the identity matrix of size N×N, and [symbol] represents the Kronecker product. The compressive sensing scheme or process [4], [5] attempts to recover the sparsest solution to X from the measurements Y using the following convex minimization:

$${argmin}_{X ∈ {R}^{d \times N}} \vert \vert {Ψ}_{MC}^{T} {X}_{vec} {\vert \vert}_{1} s . t . Y = {Φ}_{MC} 𝒫 ( X ) \qquad ( 6 )$$

where X_{vec} is the vector form of X which includes the concatenation of its columns. The l₁ norm of a vector ξ is defined as ∥ξ∥₁=Σᵢ|ξᵢ|. The measurement consistency in the recovery algorithm (Y=φ_{MC} [symbol] (X)) pertains to obtaining a signal X which is in accordance with the measurements Y through the measurement matrix φ_{MC}, i.e., (6) recovers a signal (X) which is sparse in the sparsity domain (ψ_{MC}) and satisfies the measurement consistency constraint.

### D. Mixed Norm Recovery

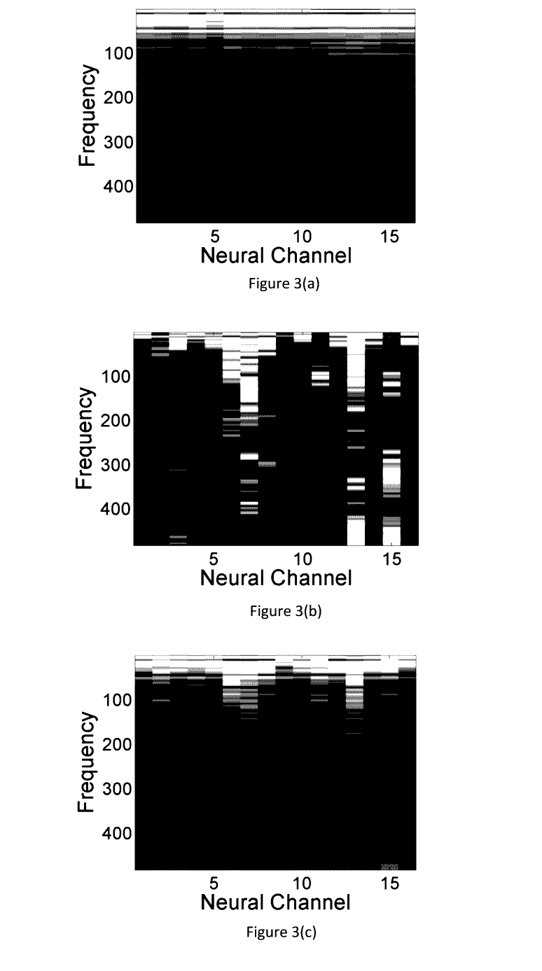

The multichannel neural signals have high inter-channel dependency, as the signals recorded by the adjacent channels are delayed or scaled version of each other, depending on the spatial resolution and pitch of the electrodes which indicates the propagation of neural activity within the brain. The dependent structure of multichannel neural signals suggests the design of a recovery model that exploits the similarity of neural signals. The Gabor coefficients [20] of iEEG signals recorded by adjacent channels in a Gaussian window are shown in FIG. 3(a). The Gabor coefficients are observed to follow similar activity among neural channels for each frequency.

Sparse recovery induces coefficient-wise sparsity without considering the inter-channel correlation of the neural signals. The neural recovery model should employ the cross correlations of iEEG signals to improve the reconstruction quality. An appropriate model for multichannel neural signals should highlight the group structure of Gabor coefficients, i.e., the model should lead to a sparsity on the number of active frequencies and promote similar activity on the neural channels for the selected frequency.

We model the dependency of neural signals using the l_{1,2} mixed norm [21]. The l_{1,2} mixed norm of a given vector ξ is defined as

$$\vert \vert ξ {\vert \vert}_{1 , 2} = {\sum }_{g} {( {\sum }_{m} \vert ξ {\vert}^{2} )}^{1 \text{/} 2} \qquad ( 7 )$$

where ξ is divided into a set of non-overlapping groups. The elements of ξ are indexed by a pair (g, m), where g defines the group number and m represents the corresponding index inside the group. It is important to differentiate between the l_{1,2} mixed norm and the sparsity inducing l₁ norm. In the former, a group of coefficients are discarded or retained together, since the same threshold is applied to the l₂ norm of each group. In the latter, each coefficient is shrunk independently from other elements and the same threshold is applied to each element. Consequently, the l₁ recovery does not model the block structure in the neural signals. However, the l_{1,2} norm respects the group structure of neural signals and imposes sparsity on the group of coefficients rather than each coefficient independently. Thus, the l_{1,2} scheme results in recovery of dense blocks for sparse number of frequencies. FIG. 3 compares the recovered Gabor coefficients of multichannel neural signal employing sparse and joint recovery in a Gabor window, with the Gabor coefficients of the original neural signal. We observe that the recovered Gabor coefficients using l_{1,2} [FIG. 3(c)] yield the same structure as of the original multichannel neural signals [FIG. 3(a)]. Furthermore, the sparse recovery [FIG. 3(b)] does not respect the group structure of neural signals and results in recovery of Gabor coefficients which are sparse and independently spread along different frequencies for each neural channel. This behavior is explained by the fact that the sparse recovery does not consider the group structure of neural signals. The solution to the multichannel neural recovery using the l_{1,2} mixed norm is obtained by replacing the l₁ norm by the mixed norm as

$${Ψ}_{MC} {argmin}_{{α}_{MC} ∈ {R}^{d \cdot N}} {‖ {α}_{MC} ‖}_{1 , 2} s . t . Y = {Φ}_{MC} 𝒫 ( {Ψ}_{MC} {α}_{MC} ) \qquad ( 8 )$$

For example, let us assume the number of channels is set to N=16 and the signal length is equal to d=1024. The exemplary 16-channel iEEG signal is mapped into a matrix XεR^{1024×16} such that the iEEG signals are along the columns of X. We define the operator [symbol] : R^{1024×16}→R^{1024.16} which transposes X and concatenates the columns of the transposed X to make a vector. The proposed circuit has a compression ratio of 4, therefore 4 measurements are recorded out of the 16 neural channels at each time. The measurement matrix is defined as:

$${Φ}_{MC} = [ \begin{matrix}{Φ}_{1} & 0 … & 0 \\ ⋮ & ⋱ & ⋮ \\ 0 & … & {Φ}_{1024}\end{matrix} ] , \\ {Φ}_{i} = [ \begin{matrix}{ϕ}_{i}^{1.1} & … & {ϕ}_{i}^{1.16} \\ ⋮ & ⋱ & ⋮ \\ {ϕ}_{i}^{4.1} & … & {ϕ}_{i}^{4.16}\end{matrix} ] , \qquad ( E1 )$$

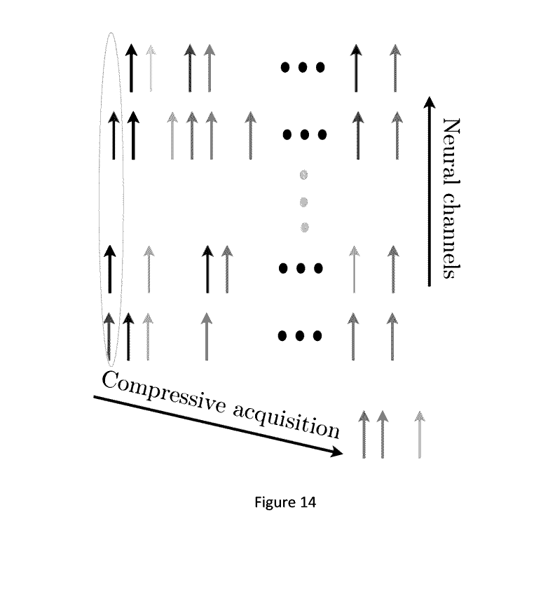

This means that at each time, the 16 channels will be multiplied by the random matrix φᵢ and result in 4 random measurements (see FIG. 14). The elements of φᵢ randomly take values in {0, 1}. The measurement vector of the multichannel neural signal, YεR^{1024.4} is computed as:

Y=φ_{MC} [symbol] (X) (E2)

The underlying neural signals in channels share similar structures. Hence, the transform domain of the multichannel signal for each neural channel is the same, that is, the sparsity of the multichannel signal reads:

$${Ψ}_{MC} = [ \begin{matrix}{Ψ}_{1} & 0 … & 0 \\ ⋮ & ⋱ & ⋮ \\ 0 & … & {Ψ}_{16}\end{matrix} ] \qquad ( E3 )$$

where ψ is the sparsity of each neural channel. We take ψ as a Gabor transform. In addition, the multichannel neural signals have high inter-channel dependency, as the signals recorded by the adjacent channels are delayed or scaled version of each other, depending on the spatial resolution and pitch of the electrodes which indicates the propagation, for example, of neural activity within the brain. The dependent structure of multichannel neural signals suggests the design of a recovery model which exploits the similarity of neural signals. The Gabor coefficients of iEEG signals recorded by adjacent channels in a Gaussian window are shown in FIG. 3(a). The Gabor coefficients are observed to follow similar activity among neural channels for each frequency. Thus, the neural recovery model should employ the cross correlations of iEEG signals to improve the reconstruction quality.

An appropriate model for the multichannel neural signals should highlight the correlated structure of Gabor coefficients, i.e., the model should lead to a sparsity on the number of active frequencies and promote similar activity on the neural channels for the selected frequency (see FIG. 3(a)). The present invention uses a scheme to model such dependent structure, which is the mixed l_{1,2} norm and divides the signal into groups such that the elements inside each group have correlated structures. FIG. 3(c) shows the recovered Gabor coefficients using the mixed l_{1,2} norm, which yields that the correlated structure of the original multichannel neural signals is well preserved.

The recovered biological signals reconstructed from the compressed measurements and solution to the multichannel neural recovery using the l_{1,2} mixed norm is obtained by:

$$\hat{X} = {{Ψ}_{MC}}_{{α}_{MC} ∈ {R}^{1024 \cdot 16}} argmin {‖ {α}_{MC} ‖}_{1 , 2} s . t . Y = {Φ}_{MC} 𝒫 ( {Ψ}_{MC} {α}_{MC} ) . \qquad ( E4 )$$

Alternatively, we do not impose the dependencies in data, that is, we instead impose the sparsity on the representation of the neural signals in the multichannel Gabor transform ψ_{MC.} Instead of the preferred l_{1,2} scheme, the recovery process can alternatively be carried out using the l₁ norm.

The sparsity constraint recovers a signal from the compressive measurements under the assumption that the signal can be well represented by a few number of dominant coefficients. Under the RIP condition on the measurement matrix, the sparsity constraint is modeled by the l₁ recovery which looks for a few entries (sparse solution) of a signal. Therefore, the sparse recovery of neural signals from the compressive measurements reads

$$\hat{X} = {argmin}_{X ∈ {R}^{d \times N}} {‖ {Ψ}_{MC}^{T} {X}_{vec} ‖}_{1} s . t . Y = {Φ}_{MC} 𝒫 ( X ) \qquad ( E5 )$$

where X_{vec} is the column-vector form of X which is the concatenation of its columns. In Equation (E5), the l₁ norm (∥•∥₁) promotes a sparse representation of the neural signals. That is the recovered neural signals should have a sparse representation in the Gabor transform, i.e., α_{MC}=ψ_{MC}^{T}X_{vec} is sparse. In addition, the measurement consistency in the recovery algorithm (Y=φ_{MC} [symbol] (X)) pertains to obtaining a signal X which is in accordance with the measurements Y through the measurement matrix φ_{MC}. Therefore, Equation (E5) looks for a signal X which is sparse in the Gabor transform and satisfies the measurements received from the decoder.

As explained, the sparse assumption on the neural signals promotes component wise sparse solution and cannot encode information about the patterns of neural coefficients in the Gabor transform. As shown in FIG. 3, it is clearly evident that the significant coefficients are organized along horizontal lines. Such structure cannot be modeled using the coefficient-wise sparsity. Therefore, we can improve the reconstruction performance of the sparse recovery by fusing this knowledge into the recovery model.

The natural way to incorporate the pattern of the signals in the sparse model is to group the coefficients into blocks such that the coefficients within a group are compared together for selection or setting to zero. In order to index the coefficients and their corresponding groups, each coefficient is indexed by a pair of (g, m), where g is the group index and m is the index of the coefficients within group g. The mixed l_{1,2} norm leads variables in the same group to be jointly selected or set to zero. To model the correlation in neural signals, the representation of neural signals in Gabor transform are grouped, where the groups are Gabor coefficients per frequency bin of Gabor transform (horizontal lines in FIG. 3). The mixed l_{1,2} norm recovery is expressed by previous Equation (E4) where ∥•∥₂ models group sparsity.

Basically this approach groups the representation of the neural signal in the Gabor transform (α_{MC}) along each frequency bin to impose the specific pattern of the Gabor coefficients. Similar to the sparse recovery, the measurement consistency guarantees that the recovered neural signal agrees with measurements acquired by the encoder. It should be noted that, in the mixed norm, recovery ψ_{MC}α_{MC} is used instead of X in the measurement consistency term. The reason is that the mixed norm promotes the pattern of the representation of the neural signals in the Gabor transform, i.e. α_{MC}, which means it recovers the representation of the neural signals in the Gabor transform. However, the sparse recovery directly looks for the neural signal X and promotes its sparsity in the Gabor transform. In the mixed norm recovery, the neural signal is recovered by \hat{X}=ψ_{MC}\hat{α}_{MC}, where \hat{α}_{MC} is the solution to the mixed norm minimization problem.

### III. Circuit Implementation

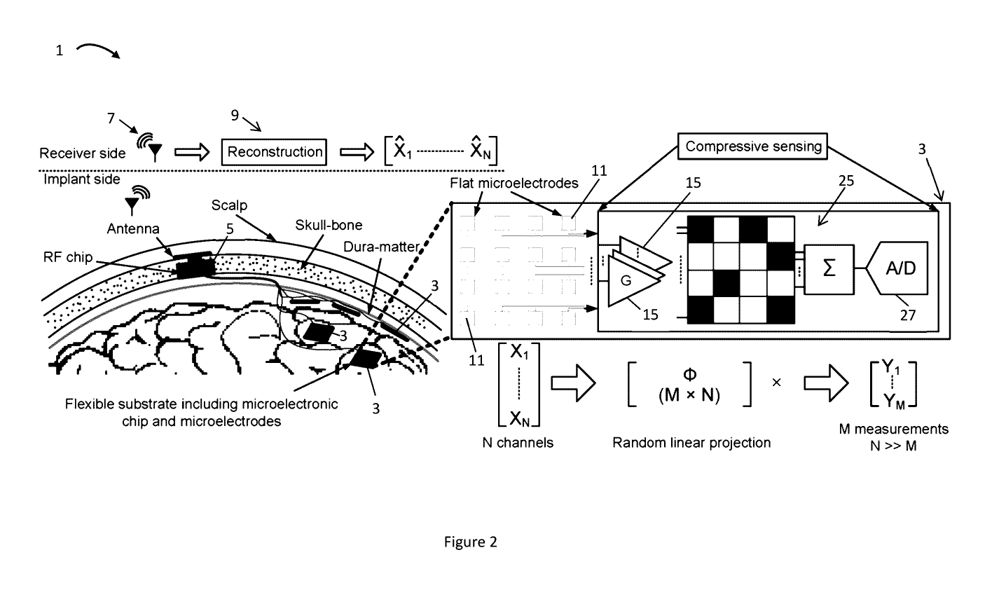

FIG. 2 illustrates an exemplary system-level view of the proposed multichannel compressive sensing method used in an implantable neural recording interface, where the neural signals sensed at the electrode sites are amplified, randomly projected, summed up and digitized through a single on-chip ADC. An RF unit placed within a burr hole in the skull transmits the compressed and digitized data originating from several recording chips to an external receiver and powers the implanted system.

FIG. 2 illustrates a system 1 for compressing a plurality of biological signals and for recovering biological signals. The system 1 includes a plurality of implantable sensors (or devices) 3, an implantable transmitter 5, a receiver 7 and a processor 9.

The implantable sensor 3 includes a plurality of electrodes 11 each of which is configured to capture or measure a biological signal, as well as a plurality of amplifiers 15. Each amplifier 15 comprises a low-noise amplifier with a band-pass transfer function and is connected to a single electrode 11 to amplify the signal captured by that electrode 11.

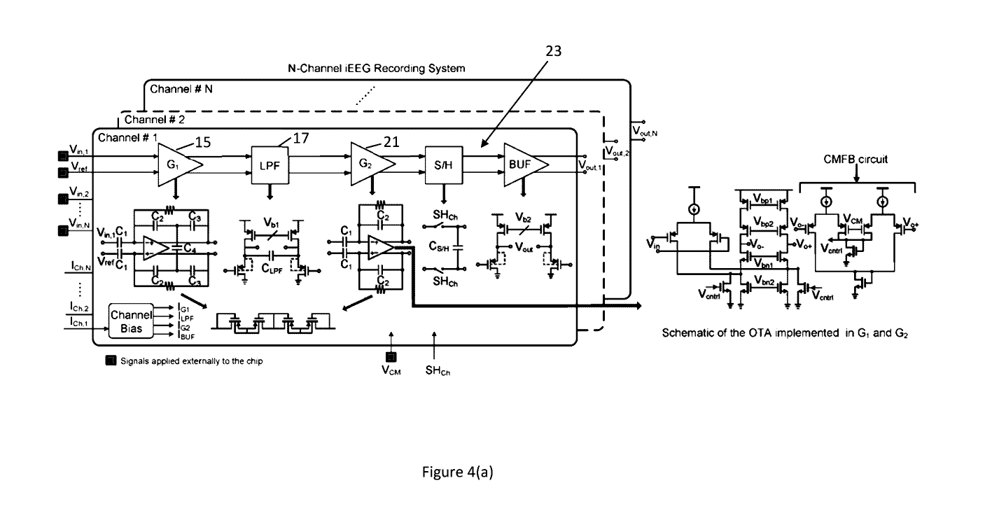

The implantable sensor 3 further includes a plurality of low-pass filters 17 (see FIG. 4) to limit the high cut-off frequency of the amplified captured signal. Each low-pass filter 17 is connected to a single amplifier 15 to receive the amplified captured signal.

The implantable sensor further includes a plurality of additional amplifiers 21. Each additional amplifier 21 is connected to a single low-pass filter 17 to further amplify the signal provided by said low-pass filter 17.

The implantable sensor additionally includes a plurality of buffered sample-and-hold circuits 23. Each buffered sample-and-hold circuit 23 is connected to a single additional amplifier 21 to receive the signal amplified by said additional amplifier 21.

The implantable sensor additionally includes a random summing circuit 25 connected to receive each of the signals provided by the electrodes 11 that each have been processed by an amplifier 15, a low-pass filter 17, an additional amplifier 21 and a buffered sample-and-hold circuit 23.

The random summing circuit 25 is configured to randomly select samples of these processed signals and sum these samples to provide a multichannel measurement signal.

The implantable sensor 3 further includes a single analog-to-digital converter 27 connected to the random summing circuit 25 to receive the multichannel measurement signal. The analog-to-digital converter 27 is configured to digitize the multichannel measurement signal to output a digital compressed signal. The compressed signal is provided to the implantable transmitter 5.

The implantable transmitter 5 includes an RF circuit and an antenna and is configured to wirelessly transmit the compressed signal to the receiver 7. The implantable RF chip 5 is further configured to provide energy to the implantable sensors 3.

The receiver 7 includes an RF circuit and an antenna to receive and process the transmitted data. The processor 9 is configured to receive the compressed signal from the receiver 7 and to process the compressed signal to reconstruct the captured biological signals.

In order to realize the CS acquisition scheme proposed in Section II, the architecture shown in FIGS. 2 and 4 is designed and implemented in a 1P6M 0.18 μm CMOS technology. The main focus of this design consists of accommodating a large number of recording units into the available die area, while preserving sufficiently low noise and low-power performance. Low power consumption and compact area are crucial in high-density implantable recording systems.

### A. Recording Channels

The fabricated integrated circuit includes 16 recording channels. Each channel consists of a low-noise amplifier with a band-pass transfer function 15, an additional low-pass filter 17 to limit the high cut-off frequency, a second gain stage 21, and a buffered sample-and-hold circuit 23. The differential outputs of all channels individually connect to the summing stage and randomly accumulate at the output of this stage. The result is then digitized through a single ADC 27.

As described above in Section II and shown in the exemplary embodiment of FIG. 1, every set of 16 channels of a pre-determined time-window (time sample) are multiplied by a random matrix φᵢ of size 4×16, which generates the four corresponding measurements. This operation then occurs within 1024 time samples over the array. Each row of the matrix is multiplied by the same neural signal vector, necessitating a holding operation at the output of the channels. Following the amplification and filtering in individual channels, the sampled and held differential outputs of every 16 pixels connect to the summing stage 25 (FIG. 4(b)) and randomly accumulate at the output of this stage 25. The outputs are controlled by the random sequences which are specific to each channel. Multi-output sequence generation is achieved by XORing the multiple outputs of maximal-length pseudo-random binary sequence (PRBS) generators (FIG. 4(c)) as will be explained in further detail below.

Depending on the instantaneous value of the random sequence, the output of the channel is summed at the output of the summing stage 25. For instance, assuming a smaller number of 4 channels for the sake of simplicity, and for φ_{R1}=1, φ_{R2}=0, φ_{R3}=1, and φ_{R4}=1, the output of the summing stage is calculated as:

V_{summed}=V_{out,1}+V_{out,3}+V_{out,4} (E6)

This summation is equivalent to the matrix multiplication presented in Section II. In total, since only 4 measurements are collected from 16 channels, a compression ratio of 4 is achieved. A variable voltage gain in the summation stage 25 is enabled through the controlling switches in series with the feedback capacitors. Due to the randomized summation at this stage, the programmability of the gain is crucial. The gain stage 29 preceding the ADC 27 provides an additional gain of three, to perfectly accommodate the full-scale input range of the ADC 27.

A three-stage configuration is used in each channel [22], in order to minimize the total die area and provide the desired amplification and filtering of the input signal.

### 1) Low-Noise Amplifier 15:

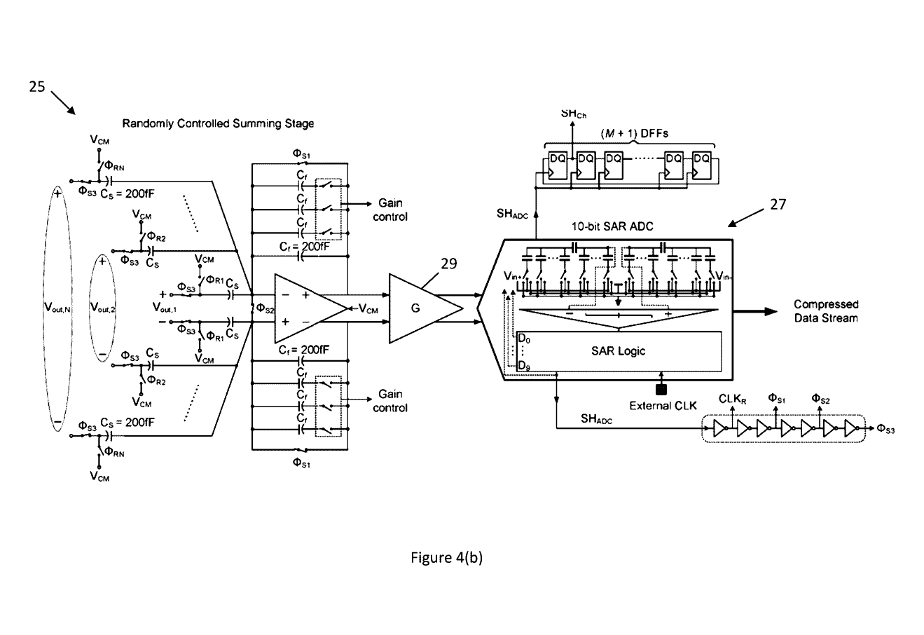

An area-efficient T network-based capacitive feedback amplifier [23] with moderate-sized input capacitors is used as the front-end gain stage G₁, which provides a mid-band gain of 29.8 dB [FIG. 5(a)]. The mid-band gain of this stage is realized by multiplying two capacitive ratios. This potentially provides a high closed-loop gain in a single stage. Using this topology, the total size of the capacitors is smaller than the size of capacitors in a conventional capacitive feedback topology [24]. Four back-to-back MOS devices biased in subthreshold region are used to implement the high-value feedback resistors with sufficient linearity for creating a low-frequency high-pass pole [FIG. 4(a)]. The variation on the high-value resistance over the voltage swing across the resistor is 1.5% at 200 mV and remains smaller than 2% up to a voltage swing of 600 mV across the resistor. Owing to the series connection of the high-value resistors in a symmetric combination, the equivalent resistance exhibits symmetric variations around the quiescent point in all process corners. The simulated high-pass cut-off frequency varies in the range of 14-118 Hz, under the worst-case process corner, supply voltage and temperature variations.

The differential mid-band gain of this stage is calculated as

$${A}_{M} = - ( \frac{{C}_{1}}{{C}_{2}} ) ( \frac{2 {C}_{4} + {C}_{2} + {C}_{3}}{{C}_{3}} ) \qquad ( 9 )$$

In general, the smaller the input capacitance in the feedback loop is, the larger the ratio of the OTA (operational transconductance amplifier) noise referred to the input of the LNA will be, since the OTA's input parasitics become comparable to C₁. To tackle this issue, the additional gain provided by the second capacitive ratio is selected small compared to the first term in (9) to satisfy the low-noise operation as well. In this design, C₂=C₃=200 fF, C₁=1.4 pF and C₄=400 fF, resulting in Cₜₒₜ=4 pF. The conventional topology [24] requires a total capacitance of 64×200 fF≈12.8 pF to achieve a similar mid-band gain.

A second benefit of replacing the very large input capacitance by a moderate one is that the larger input impedance provided by a moderate input capacitor reduces the effect of attenuation of cortical signals in the electrode-channel interface.

A folded-cascode OTA with a continuous-time common-mode feedback (CMFB) circuit is implemented in the LNA [FIG. 4(a)]. Through the dc path provided by the large-value feedback resistors, the same CM voltage as V_{CM} is generated at the input gates of the OTAs used in G₁ and G₂. The total bias current of the OTA is 7 μA. The simulated passband of the LNA ranges from 28 Hz to 144 kHz. The linearity performance of the LNA is described by the THD metrics which is 0.24% for a 2 mV input signal. This LNA consumes 8.4 μW of power, corresponding to 89% of the total power consumed by the channel.

The ac-coupled architecture provided by the capacitive feedback topology enables the amplifier to reject the large dc offsets (as large as 1-2 V [24]) which are commonly generated in an electrode-tissue interface. The common centroid layout of the input differential pair in the LNA and the passive components such as input and feedback capacitors results in improved matching and offset performances, which are mainly limited by the front-end LNA. The measured input-referred offset of the stand-alone channel is 0.85 μV.

### 2) Filter 17, Amplifier 21 and S/H 23:

The commonly used low bandwidth of interest (less than 2 kHz) for capturing iEEG signals (e.g., for epileptic activity detection) requires additional low-pass filtering after the LNA. Limiting the bandwidth using a low-pass filter stage with minimal power and noise costs is more beneficial to limit the die area per channel than aggressively increasing the C_{L} of the LNA. A first-order source-follower based low-pass filter (adapted from [25]) is used which achieves sufficient linearity at a small bias current [FIG. 4(a)]. The DC-gain loss due to the bulk transconductance which is inherent to the source-follower architecture is reduced by a source-to-bulk connection. The internal feedback of the source-follower circuit and processing the signal in voltage domain increases the linearity range of the filter.

The presented filter exhibits a THD of 0.08% (equal to 10-11 bits of linearity) for a filter input signal of 60 mVₚ₋ₚ (or 2 mV at the input of a channel) consuming a bias current of 5.4 nA. The cut-off frequency is 1.9 kHz and is tunable by the current.

A second gain stage 21 [G₂ in FIG. 4(a)] provides an additional gain of dB using a conventional capacitive feedback architecture. The power consumption of the second gain stage is negligible (0.72 μW) compared to the front-end LNA, thanks to the relaxed noise requirements. Using a folded-cascode OTA architecture, an output voltage swing as large as 200 mVₚ₋ₚ is achieved in the second gain stage. The size of the transistors in this OTA are selected smaller compared to the OTA used in the front-end LNA, thanks to the relaxed noise and matching requirements.

The amplified and band-pass filtered signal of each channel goes through a sample-and-hold (S/H) stage 23 followed by a source-follower buffer to keep the signal constant during the consecutive M measurements (M=N/CR in this case). The S/H circuit is controlled by the signal SH_{Ch} which is (M+1) times slower than the signal of the ADC and is generated using a single (M+1)-bit shift register [FIG. 4(b)]. The output common-mode voltages of the LPF and BUF are defined by the gate-source voltage of the PMOS transistors which are located at their input, knowing that the output bias voltage of the preceding stages are defined through the CMFB of G₁ and G₂. The source-to-bulk connected source-follower buffers at the output of each channel exhibit a THD of 0.12%, which equals to 9-10 bits of linearity for a 200 mVₚ₋ₚ input signal. The total power consumption of the LPF, buffer and in-channel biasing circuitry is less than 0.3 μW.

The required settling time to guarantee B-bit performance imposes the following condition at the output of the second gain stage, prior to the S/H:

$${e}^{- ( \frac{1 / ( 2 fs )}{τ} ) < 0.5 \times {2}^{- B}} \qquad ( 10 )$$

for B=8 and f_{S}5=4 kS/s this condition results in

$${𝒻}_{- 3 d B} = 1 / 2 π τ > \frac{( B + 1 ) ln 2}{π} {𝒻}_{s} \approx 1.98 {𝒻}_{s} \approx 8 kHz \qquad ( 11 )$$

in which f_{-3 dB} represents the bandwidth of the circuit when the sampling capacitor is connected. Assuming a sampling capacitor of 0.4 pF, the achieved bandwidth is 55 kHz, consuming a low bias current of 0.6 μA.

### B. Randomly Controlled Summing Stage 25

The sampled signals of channels of the array connect to the summing stage at the sampling phase (φ_{S3}) which follows the two in-phase events φ_{S1} and φ_{S2}. By applying a proper timing strategy φ_{S1} comes before φ_{S2} and φ_{S2} before φ_{S3}) as presented in FIG. 4(b) and using bottom-plate sampling, it is possible to substantially reduce the effect of channel charge injection of the switches. The effect of clock feedthrough is reduced by the differential implementation. A variable voltage gain is achieved through the controlling switches in series with the feedback capacitors. Due to the randomized summation at this stage, the programmability of the gain is crucial. The gain stage preceding the ADC 27 provides an additional gain of three to perfectly accommodate the full-scale input range of the ADC 27. A hybrid two-stage class A/AB topology [28] is used as the OTA in this stage, which provides the desired rail-to-rail output swing.

The operation of the summing stage is as follows. In sampling mode, φ_{S1}, φ_{S2} and φ_{S3} are on, allowing the differential voltage across the two sampling capacitors (C_{S}) at the output of each channel to track the differential output voltage of that channel. In summation mode (φ_{S1}, φ_{S2} and φ_{S3} are off), the charge stored on the sampling capacitors of those channels with random value equal to one are transferred to the capacitors in the feedback path (C_{f}). This enables the summation of the sampled values of channels, based on the value of the corresponding random sequence.

A reasonable lower bound for the bandwidth of OTA used in the summing stage can be calculated from (10) which equals 50 kHz at a sampling rate of (M+1) f_{S}=20 kS/s. The stability and speed of the feedback loop is guaranteed for the worst case where all random values are equal to one, while a single feedback capacitor is connected which corresponds to A_{M,INT}=N, for equal unit capacitors and equal inputs.

### 1) System Resolution:

Assuming the worst-case situation in which all channels of the array are summed together while all N controlling random values are equal to one, the following constraints on the signal headroom and noise prior to the ADC will exist:

NV_{in,sig}A_{M}≦2VDD (12)

NV_{n,rms}²A_{M}²≦(2VDD)²/12·2^{2By} (13)

where A_{M} is the total mid-band gain from the input of the channels to the input of the ADC, V_{in,sig} is the peak-to-peak amplitude of the input signal, V_{n,rms} is the rms value of the input-referred noise of each channel and N is the number of channels.

Having

$${V}_{in , rms} = \frac{{V}_{in , sig}}{2 \sqrt{2}}$$

for a sinusoidal waveform, results in B_{y}≈B_{sig}+log₂\sqrt{N}−0.58. Generalizing this equation to any arbitrary waveform with

$${V}_{in , rms} = \frac{{V}_{in , sig}}{K}$$

yields

$${B}_{y} \approx {log}_{2} \frac{{V}_{in , sig}}{{V}_{n , rms}} + {log}_{2} \sqrt{N} - 1.78 \qquad ( 14 )$$

which is independent of the parameter K. Considering a background neuronal noise of 5-10 μVᵣₘₛ [26] and a typical amplitude of surface cortical signals of up to 1 mV and adding some extra margin, B_{sig} and B_{y} are set to 8 and 10 for 16 channels. This calculation excludes the rms thermal noise of the electrodes which can be in the order of 10-20 μV [27], depending on the size, material and operating temperature of the electrodes. In reality, this noise further degrades the dynamic range of the input signal and the effective number of bits achievable at the system level.

The effective compression ratio of the proposed CS topology is then approximated by

$${CR}_{eff} \approx \frac{{NB}_{sig}}{( M + 1 ) ( {B}_{sig} + {log}_{2} \sqrt{N} )} \qquad ( 15 )$$

### 2) Large-Scale Implementation:

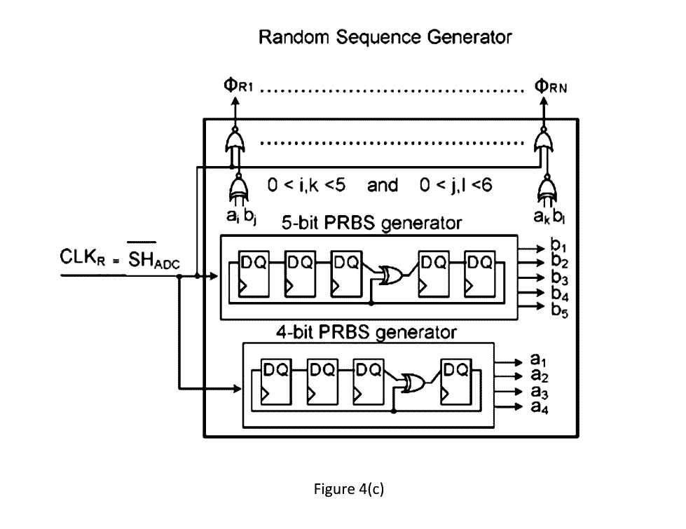

The imposed requirement on the ADC resolution degrades the power efficiency of the system at large number of channels and sets an upper limit for the maximum number of channels connected to the summing stage prior to the ADC. Thus, the model shown in FIG. 6 is adapted for large arrays. The digital outputs of 16-channel blocks are summed together by a digital accumulator with sufficiently large number of bits. The power and area cost of adding the required number of bits to the resolution of the accumulator is significantly less compared to the cost of increasing the resolution of the ADC. Designing an ADC for target resolutions higher than 10 bits requires additional topological modifications which are commonly achievable by consuming more power. Using this method, the proposed CS concept is still more area- and power-efficient for large arrays than single-channel approaches.

In this model, the limit on the compressed array size is imposed by the acceptable power efficiency of the summing stage and ADC which are sampled at a rate of

$$( 1 + \frac{N}{CR} ) {𝒻}_{s}$$

with f_{S} being the Nyquist sampling rate. The multichannel system is then divided into sub-blocks of N-channels, with N being the optimum number of channels to be compressed into a single data stream. Each sub-block is encoded to a digital data stream which after reconstruction, generates the signals originated from the channels constituting that sub-block.

### 3) Random Matrix Generator:

In order to guarantee an efficient implementation of compressive sensing, the power and area cost of CS circuits including the measurement matrix generator must be negligible compared to the rest of the integrated circuit. In a single-channel approach, each channel is loaded with m sequences (building the rows of the measurement matrix explained in (1)), whereas in the proposed model, each channel needs to be driven by only one sequence. As shown in (3), the measurement matrix supporting the consecutive M measurements for recovering each sample of individual channels is filled by the corresponding M values of the in-channel sequences.

This approach circumvents the need to place a memory to store the elements of the measurement matrix.

As shown in FIG. 4(c), multi-output sequence generation is achieved by XORing the multiple outputs of maximal-length pseudo-random binary sequence (PRBS) generators [29]. For a recording array of 4×4 and a value of M equal to 4 (resulting in CR=16/4), the 16 sequences driving the individual channels are generated by XORing the states of a 4-bit PRBS generator with another 5-bit PRBS generator. As opposed to the single-channel compressing system which has to be physically designed for a specific predefined and redesigned by varying the compression ratio, the proposed scheme is easily adaptable for different values of M by adjusting the clock frequency.

### 4) Noise Analysis:

Power, area and noise are the three major performance metrics of an implantable system. Ideally, the noise performance of the system is limited by the background extracellular noise and by the noise of the electrodes. In addition to the low-noise amplifier at the front-end of each channel, the noise induced by the switched capacitor summing stage should be minimized. While the effect of flicker noise can be reduced by using large input devices (generally PMOS), the thermal noise induced by the switching circuits can affect the total noise of the system, prior to the ADC. A comprehensive sampled noise analysis based on an approach similar to [30] is presented to give an insight into the appropriate values of the design parameters of the summing stage. As a general noise reduction technique, the input differential pair of the folded-cascode OTA used in the front-end LNA is biased in subthreshold region [31].

This results in a large gₘ/I_{D} of the input transistors and reduces the input-referred noise of the OTA. The mean-square value of the total thermal noise at the output of each channel can be approximated by integrating the PSD of OTA noise at the input [31], shaped by the low-pass transfer function of the loop [see the half-circuit model shown in FIG. 5(b)], and fed into the low-pass filter and second gain stage, resulting in

$$\overline{{v}_{n , out}^{2}} \approx {S}_{n , op} \cdot \frac{{A}_{M , tot}^{2}}{4 τ} = \frac{4 kT}{κ {g}_{m}} \cdot \frac{{A}_{M , tot}^{2}}{4 τ} \qquad ( 16 )$$

where k is Boltzmann's constant, T is the absolute temperature, κ is the reciprocal of the subthreshold slope factor,

$${A}_{M , tot} \approx \frac{1}{{β}_{1} {β}_{2}} ,$$

1/β₁ is obtained from (9) and 1/β₂ is the mid-band gain of the second gain stage. Assuming

$${τ}_{1} = \frac{{C}_{L}}{{β}_{1} {g}_{m}}$$

the settling time constant at the output of the LNA with a load capacitor of C_{L} and an input transconductance of gₘ, and

$${τ}_{2} = \frac{{C}_{LPF}}{{g}_{m , LPF}}$$

the time constant at the output of the LPF, τ₂>τᵢ and (16) yields

$$\overline{{v}_{n , out}^{2}} \approx \frac{kT \cdot {A}_{M , tot}^{2} \cdot {g}_{m , LPF}}{κ {g}_{m} {C}_{LPF}} \qquad ( 17 )$$

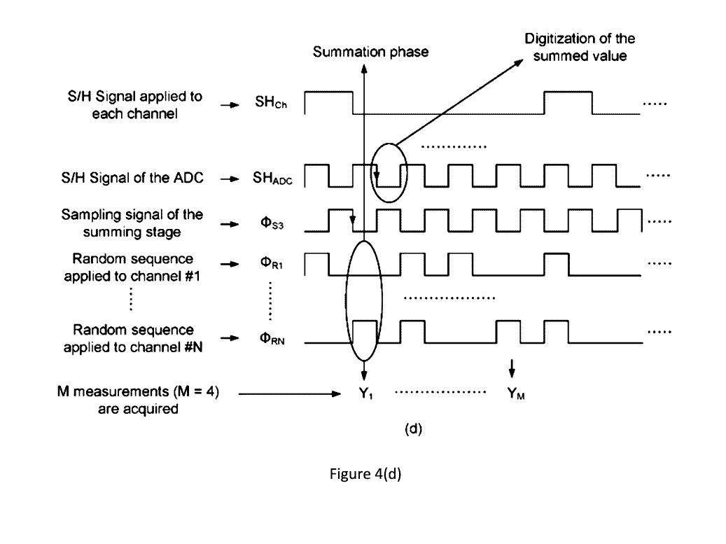

The equivalent half-circuits of the summing stage during the sampling and integrating phases are shown in FIG. 7. The PSD of the thermal noise generated on the sampling capacitor (C_{S}) which is due to the switch placed in-series with C_{S} and during φ_{S3} can be written as [30]

$${S}_{v} ( f ) = \frac{4 {kTR}_{on}}{1 + {( 2 π f {τ}_{0} )}^{2}} \qquad ( 18 )$$

Here, Rₒₙ the on resistance of the switch and τ₀ is the time constant of the C_{S} branch during sampling (φ_{S3}), which is expressed as

$${τ}_{0} \approx {C}_{S} \cdot ( {R}_{on} + \frac{{R}_{on}}{2 + {g}_{m} {R}_{on}} ) \qquad ( 19 )$$

The corresponding noise integrated from 0 to

$$\frac{kT}{[ {C}_{S} ( 1 + \frac{1}{2 + {g}_{m} {R}_{on}} ) ]}$$

which summed up to the non-significant noise terms generated by the two switches φ_{S1} and φ_{S2} can be estimated as kT/C_{S}.

During the second phase (φ_{S3} is open), the equivalent time constant of this circuit can be approximated by

$$τ = {C}_{S} ( {R}_{on} + \frac{1}{{g}_{m}} ) .$$

The mean-square value of the switching noise assuming that in the worst case, φ_{Ri} is equal to one for i=1, . . . , N, is calculated as [FIG. 7(b)]

$$\overline{{v}_{n 1}^{2}} \approx \frac{4 {kTR}_{on}}{4 τ} = \frac{kT / {C}_{S}}{1 + 1 / x} \qquad ( 20 )$$

The parameter x is introduced as x=Rₒₙ gₘ. The noise power in C_{S} due to the op-amp when φ_{S3} is open is found as

$$\overline{{v}_{n 2}^{2}} \approx \frac{4 kT / κ {g}_{m}}{4 τ} = \frac{kT}{κ {C}_{S} ( 1 + x )} \qquad ( 21 )$$

Thus, the total input-referred noise of the differential summing stage can be expressed as

$$\overline{{v}_{n , m}^{2}} \approx \frac{2 kT}{{C}_{S}} + \frac{2 kT / {C}_{S}}{1 + 1 / 1 x} + \frac{2 kT}{κ {C}_{S} ( 1 + x )} = \frac{kT}{{C}_{S}} ( \frac{4 x + 2 + 2 / κ}{x + 1} ) = \frac{kT}{τ {g}_{m}} ( 4 x + 2 + 2 / κ ) \qquad ( 22 )$$

referred to the input of each summing branch (or equivalently to the output of each channel). Based on this equation, the noise is minimized for x<<1. The total noise power calculated in (22) should be smaller than the output noise power of channels calculated by (17) which satisfies the noise requirement prior to the ADC 27. There is a clear trade-off between the summing stage's noise and the circuit area which is proportional to C_{S}. However, considering the extra margin assumed in calculating the system resolution based on (14), and accounting for the electrode and background noise, enables letting the noise due to this stage be as large as the noise of channels, keeping in mind that additional noise reduction does not improve the effective number of bits but further degrades the area efficiency of the implantable system. The typical value of the in-band input-referred noise power due to the LNA is more than 10 times smaller than the electrode noise power for this application.

With this assumption, a minimum value of C_{S} equal to 200 fF is required to guarantee the proper performance of the summing stage. For an optimal design, the maximum permissible value of τ based on (10) can be substituted into

$$τ = \frac{{C}_{S} ( 1 + x )}{{g}_{m}}$$

resulting in the lowest gₘ which can fulfill the noise and bandwidth requirements of this stage. The assumed constraint on (i.e., x<<1) determines the minimum size of the switches in this case. For equally-sized capacitors, these switches are selected (M+1) times larger than the in-channel S/H switches which operate at a lower frequency.

### C. Analog-to-Digital Converter 27

Following the random summation and an additional amplification, the random accumulated value is digitized using a single analog to digital converter 27 (FIG. 4b). The main advantage of the proposed topology is that, as opposed to conventional CS architectures, the random accumulation and ADC 27 are implemented only once per 16 channels, resulting in improved power and area efficiency of the system.

The stringent area and power constraints of the implantable system motivate the compact and energy-efficient implementation of the ADC 27. Based on system-level requirements, an ADC with 10 bits of resolution and a sampling rate of 20 kS/s translates into a data recorded in each channel with 8 bits of resolution and a sampling rate of 4 kS/s. The SAR ADC is a popular architecture which enables low-power data conversion for medium resolution/speed applications. A binary-weighted capacitive array with attenuation capacitor [FIG. 4(b)] is used which enables the compact implementation of the ADC. The potentially small input capacitance of this array results in relaxed specifications of the stage preceding the ADC, in terms of bandwidth and power consumption. The total required capacitance of the attenuated array is times smaller than a conventional binary-weighted array where is the number of bits.

As shown in the timing diagram of FIG. 4(d), the data sampled from a channel is kept constant during the M randomized summations and consecutive digitizations through the ADC 27. Thanks to the low bandwidth of iEEG signals, the PRBS generator and ADC are clocked at a rate faster than the sampling frequency of the individual channels. This allows the computations to be done within the input sampling period. All the required signals are generated using a single external clock of 400 kHz, as shown in the timing diagram of FIG. 4(d). The sample-and-hold signal of the ADC is generated at a rate equal to 1/20 of the external clock, which enables 10-bit conversion of the summed value.

Digitization occurs during the half-cycle in which random sequences are zero [shown in FIG. 4(d)], thus converting the previous accumulated value. At the same time, the sampling signals of the summing stage [φ_{S3} and also the in-phase signals φ_{S1} and φ_{S2} in FIG. 4(b)] are equal to one, which resets the output of the summing stage and stores the corresponding channel's output for the following accumulation phase. During the next half-cycle, the sampled values at the input branches of the integrator which are stored on the capacitors C_{S}, are summed together based on the value of the random sequence controlling that branch. Thus, the output of the summing stage at the rising edge of the φ_{S3} represents the randomized summation of the channels' samples which must be converted to the digital bit stream, using the ADC.

### IV. Experimental Results

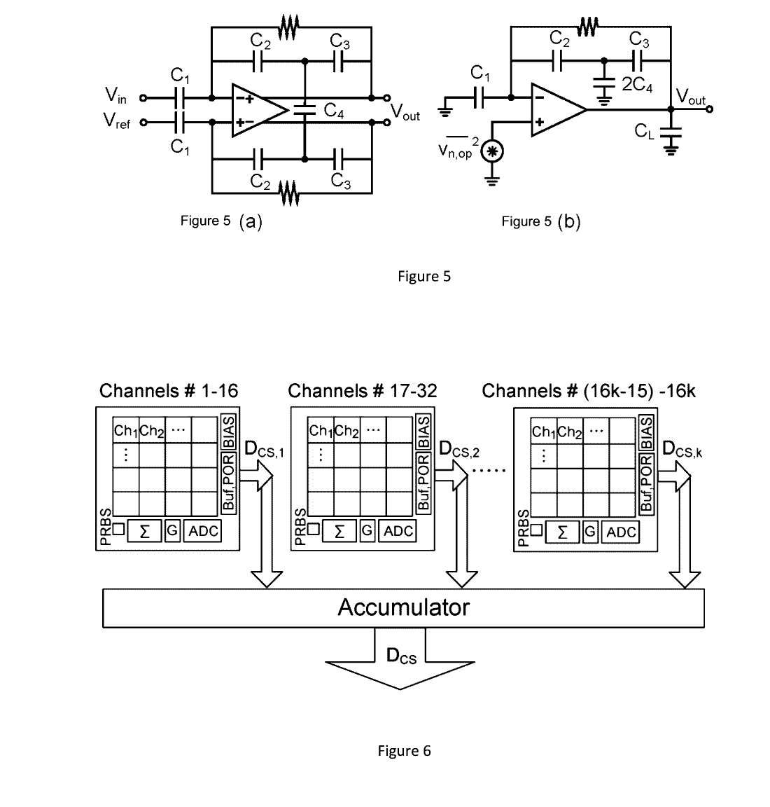

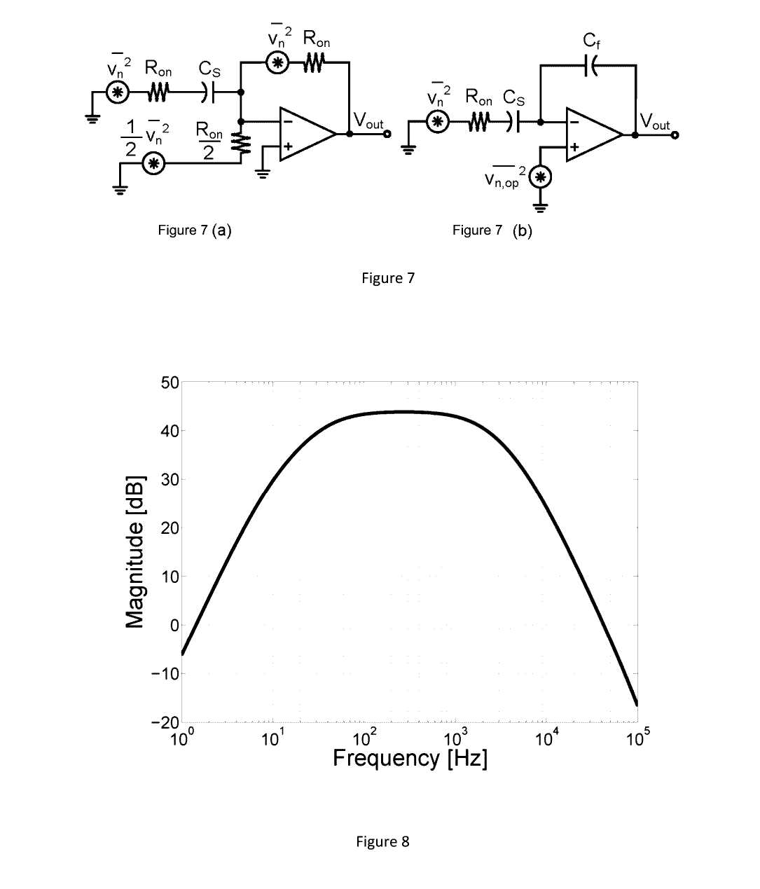

The measured frequency response at the output of G₂ and input-referred noise of the stand-alone channel are shown in FIGS. 8 and 9. The high-pass pole is measured at 39 Hz, while the simulated cut-off frequency is 28 Hz. Thus, the HP-pole differs by 40% with respect to the desired value. The discrepancy between the simulated and measured high-pass pole is due to the inaccurate model of the transistors in weak inversion region, and the extra parasitics which affect the total capacitance of the feedback path. A more precise control on the high-pass cut-off frequency can be achieved by adjusting the feedback capacitor and resistor using digital words as shown in [37].

The input-referred noise density at the channel input is 25 nV/√Hz at 1 kHz. The input-referred noise integrated over the signal bandwidth is 3.2 μVᵣₘₛ, while it increases to 4.2 μVᵣₘₛ when integrated from 1 Hz to 100 kHz. The Noise Efficiency Factor (NEF) of the analog front-end (including G₁, LPF and G₂) is defined as [32]

$$NEF = {V}_{n , rms} \cdot \sqrt{\frac{2 {I}_{tot}}{π \cdot {U}_{T} \cdot 4 kT \cdot BW}} \qquad ( 23 )$$

The measured NEF is equal to 4.12 and 3.23 for the noise integration bandwidths of 1 Hz-100 kHz and passband of the signal, respectively.

Table I presents the performance summary of the analog front-end (AFE) and ADC which are compared with published works.

TABLE I
COMPARISON OF THE AFE AND ADC PERFORMANCE WITH PUBLISHED LITERATURE

| Parameter | [38] | [39] | [37] | [40] | [41] | [42] | [23] | [43] | [44] | This Work |
|---|---|---|---|---|---|---|---|---|---|---|
| Technology [μm CMOS] | 0.35 | 0.13 | 0.13 | 0.18 | 0.35 | 0.35 | 0.35 | 0.13 | 0.13 | 0.18 |
| Bandwidth [Hz] | 10-5k | 0.023-11.5k | 167-6.9k | 10-7.2k | 1-5k | 217-7.8k | 1-8.5k | 1-5k | 0.1-5k | 39-1.9k |
| Gain [dB] | 33 | 38.3 | 47.5 | 39.4 | 34 | 45.7 | 38.1 | 54-60 | 54-60 | 43.8 |
| Input-Referred Noise [μVᵣₘₛ] | 6.08 | 1.95 | 3.8 | 3.5 | 7 | 4.43 | 13.3 | 5.1 | 6.5 | 4.2 |
| NEF | 5.55 | 2.48 | 2.16 | 3.35 | 4.6 | 2.16 | 7.87 | 4.4 | 7.2 | 4.12 |
| AFE Area [mm²] | 0.020 | — | <0.080 | 0.062 | 0.020 | — | 0.056 | 0.045 | <0.090 | 0.040 |
| CMRR | 60 | 63 | 83 | 70.1 | — | 58 | 74 | 75 | 75 | 59.8 |
| PSRR | — | 63 | — | 63.8 | — | 40 | 55 | — | — | — |
| AFE Power Consumption [μW] | 8.4 | 12.5 | 1.92 | 7.92 | 4.2 | 1.26 | 6 | 8.5 | 4.5 | 9.4 |
| ADC Sampling Fequency [kS/s] | 111 | 10-100 | 22.5 | — | — | 2.56 | — | ≦100 | 57 | 20 |
| ENOB | 6 | 6.2 | 7.65 | — | — | 8.3 | — | 7.6 | 7.8 | 9.2 |
| INL [LSBs] | — | — | −0.45 | — | — | 1.52 | — | 0.7 | — | −0.76 |
| DNL [LSBs] | — | — | −0.71 | — | — | 0.89 | — | 0.6 | — | −0.37 |
| ADC Power Consumption [μW] | 2.77 | <1 | 0.5 | — | — | 31.3 | — | 1.5 | 1.8 | 2.6 |

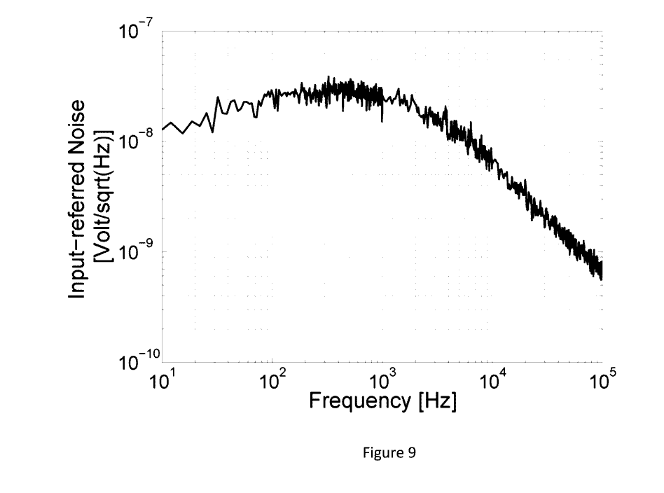

The microphotograph of the fabricated chip is shown in FIG. 10(a). The total current consumption of the chip including the buffers and bias generators is 140 μA drawn from a 1.2 V power supply, corresponding to effective current of 8.75 μA per channel. The achieved power density of the system is 7.2 mW/cm², significantly below the safety limit of 80 mW/cm² [33] for an implantable system. The contribution of different blocks of the system to the total power consumption is shown in FIG. 10(b). The area occupation and power consumption of each block of the system are shown in Table II.

TABLE II
POWER CONSUMPTION AND AREA
USAGE OF INDIVIDUAL BLOCKS

| Parameter | Value |
|---|---|
| P_{Channel} | 9.4 μW |
| P_{SummingStage} | 8.4 μW |
| P_{GainStage} | 4.2 μW |
| P_{ADC} | 2.6 μW |
| P_{PRBS} | 0.03 μW |
| A_{Channel} | 220 μm × 180 μm |
| A_{SummingStage} | 290 μm × 150 μm |
| A_{GainStage} | 150 μm × 100 μm |
| A_{ADC} | 290 μm × 150 μm |
| A_{PRBS} | 90 μm × 80 μm |

In order to demonstrate the effectiveness of the proposed acquisition model, a long segment of multichannel iEEG signal recorded from subdural strip and greed electrodes implanted on the left temporal lobe of a patient with medically refractory epilepsy have been used as the input.

The signals are recorded during an invasive pre-surgical evaluation phase to pinpoint the areas of the brain involved in seizure generation and to study the feasibility of a resection surgery. This data includes minutes of pre-ictal, ictal and post-ictal activities sampled at 32 kS/s, using Neuralynx. The signals recorded by 16 adjacent channels of a greed of the electrodes are applied into the proposed CS system.

### A. Recovery Performance

Many biological signals can be sparsely represented in either Gabor or wavelet domains [8]. We employ Gabor transform as the sparsity domain of neural signals for multichannel neural recovery based on sparse and mixed norm methods. The recovery SNR of the reconstructed signal (\hat{x}) with respect to the original signal (x) is calculated from

SNR=−20 log₁₀(∥x−\hat{x}∥₂/∥x∥₂) (24)

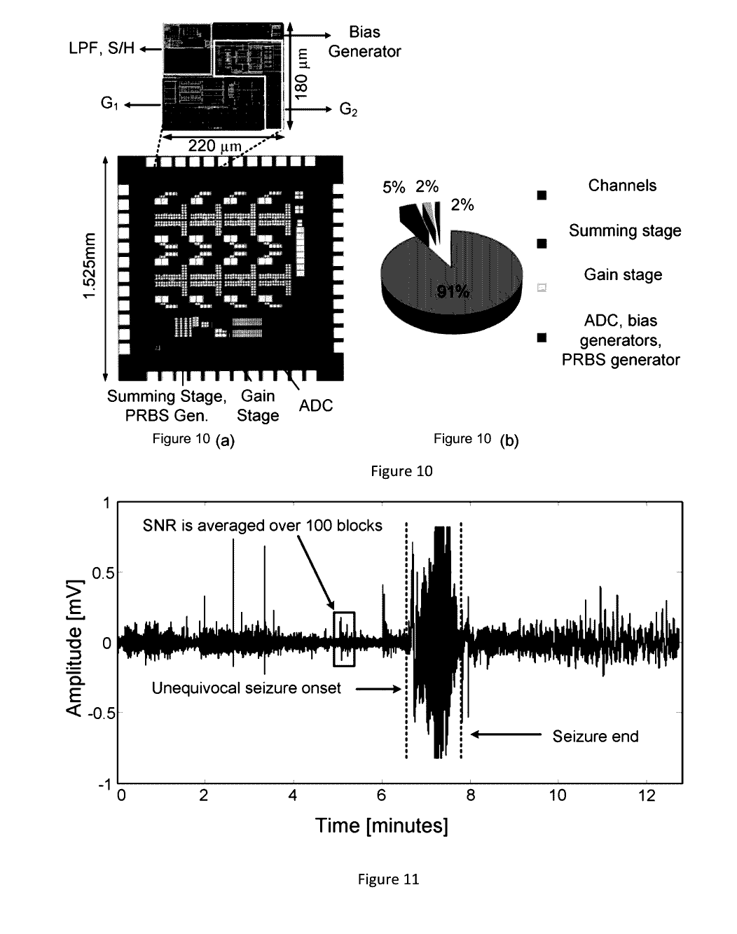

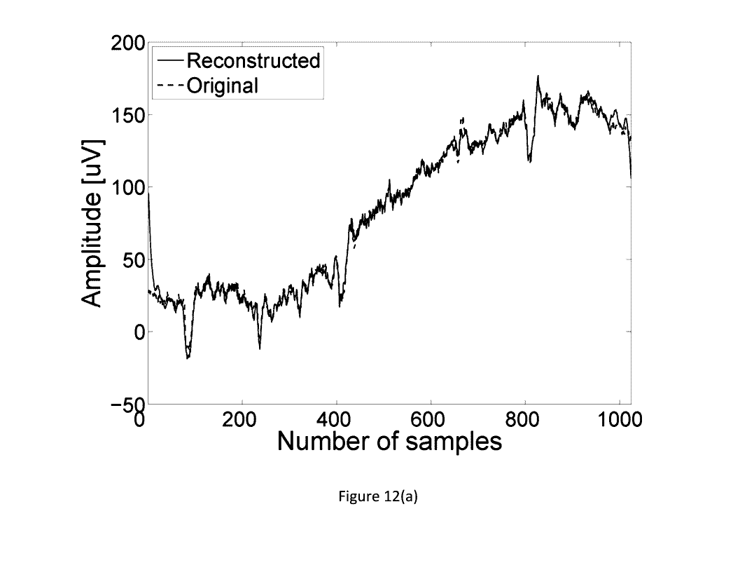

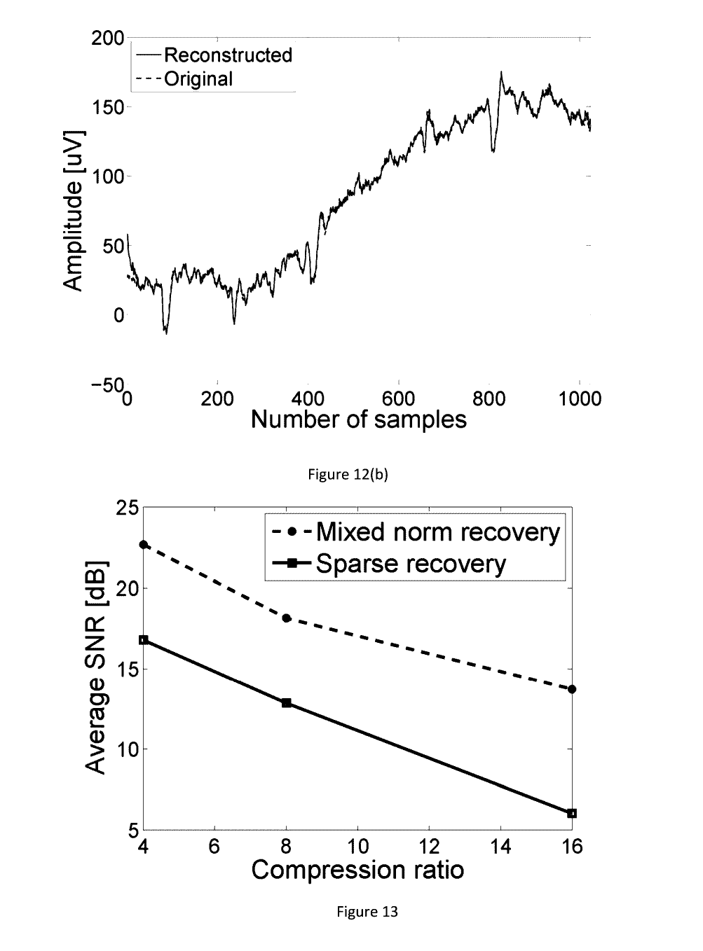

for each recording channel (e.g., represents the recovery SNR_{CH1} of first channel). The mean SNR of 16 channels are averaged over 100 blocks of the signal, as shown in FIG. 11. The performance of the circuit is validated for low-voltage fast activities which are shown to be associated with seizure onset. The reconstructed signals versus the original signals corresponding to one block of a single channel data using l₁ and l_{1,2} recovery are shown in FIGS. 12(a) and (b). The length of each compression block (d) is equal to 1024 samples and is equivalent to 256 msec at a 4 kHz sampling frequency. The digitized data after ADC is used for recovery. As shown in these figures, applying the recovery on the compressed data produced by the adjacent channels results in an improved recovery performance, compared to the sparse recovery. The averaged SNRs using the l₁ and l_{1,2} recovery are 16.64 dB and 21.80 dB, respectively. Based on the statistical analysis reported in [34], a minimum SNR of 10.45 dB (corresponding to a PRD of 30%) is acceptable to maintain the diagnostically important data in the recovered signal, e.g., for successful seizure detection. Reducing the number of measurements to M=1, i.e., CR=16 results in average SNR=13.72 dB, using l_{1,2} recovery. Thus, the system is able to successfully recover low-voltage iEEG signals compressed by a ratio as high as 16. FIG. 13 presents the average reconstruction SNR for sparse and joint recovery for different compression ratios. The achieved compression ratios vary within the range of 16/15 up to 16, corresponding to CR=16/M, where M is an integer number between 1 and 15.

As confirmed by the average SNR, the system is potentially able to recover the signal over the entire recording period, with some tolerable loss (i.e., SNR>10.45 dB). However, the amplitude and frequency of the recorded signals can significantly vary, depending on the distance between the microelectrodes and their surface area [35]. The amplitude of the very high frequency oscillations (250-500) recorded using macroelectrodes [36] is much smaller (5-30 versus 100-1250 μV) than human fast ripples recorded from smaller microelectrodes [35]. The amplitude of the fast ripples recorded by the high-density arrays will expectedly fall within the range of tens-hundreds of microvolts, which is efficiently recovered at the output of the system, as shown in FIG. 12.

The required time for the reconstruction of the 1024-length epochs of the multichannel signal is 1.12 second per channel, using a 2.66 GHz processor with 4 GB of RAM. To achieve a real-time performance, the speed of the reconstruction could be improved by hardware implementation of the algorithm on FPGA and using custom acceleration techniques.

### B. Effect of Circuit Non-Idealities and Non-Adjacent Channels

A second database consisting of multichannel intracranially recorded signals of the slices of a rat somatosensory cortex under bicuculline, which blocks the synaptic inhibition and consequently mimics the epilepsy, is applied to the CS recording system. This signal includes epileptiform burst activity and extracellularly detected spikes. However, the verifiable signals of this database are associated with electrodes randomly located on the array. Consequently, these channels exhibit limited synchronous activity during the seizure, compared to the previous database. This effect is reflected in relatively lower recovery SNR of neural signals presented in Table III. SNR is evaluated for each channel and for the multichannel signal, by comparing X_{vec} with the reconstructed multichannel data stream (defined as SNR_{T}).

TABLE III
RECOVERY QUALITY IN THE PRESENCE
OF NOISE (SNRS ARE IN DECIBELS)

| | Gabor Transform, l₁ recovery, CR = 4 | | | | |
|---|---|---|---|---|---|
| | x QN ^{a} | ✓ QN | x QN | ✓ QN | MAT |
| Performance Metric | x cktN^{b} | x cktN | ✓ cktN | ✓ cktN | LAB |
| SNR_{CH1} | 14.83 | 14.64 | 14.75 | 14.32 | 15.12 |
| SNR_{T} | 10.82 | 10.52 | 10.76 | 10.23 | 10.97 |

^{a} Excluding the quantization noise.
^{b}Excluding the noise of the circuit.

In order to study the effect of circuit non-idealities (such as quantization and thermal noise) on the recovery performance, a comparison of SNR is also presented in Table III. The reconstruction results are compared by including and excluding different noise sources in simulations. The results are compared to the recovery performance of the compressed signal generated by matrix multiplication in MATLAB, using the same matrix as the output of the on-chip PRBS generator. CS can improve the attainable signal fidelity in the presence of sensor noise as shown in [45]. Although the reconstruction performance of a CS system is not as good as a simple quantizer for noiseless inputs [46], for more practical noisy signals recorded by the sensors, CS achieves a better performance, i.e., lower energy and improved PRD (Percentage Root-Mean Squared Difference) [45].

The CS system filters some of the input noise during reconstruction [45], and consequently is a correct choice for noisy environments such as a neural interface. Otherwise expressed, the recovery algorithm discards the coefficients below a certain threshold in the sparse representation of the signal. The discarded coefficients can be interpreted as filtered noise. The recovery performance of the proposed system is marginally affected by the quantization noise of the ADC, as confirmed by the results of simulations presented in Table III.

Excluding the circuit noise in simulations (thermal and flicker) results in a negligible improvement of the recovery performance which confirms the robustness of the CS system against non-idealities induced by the circuit. Consequently, the specifications related to the resolution of the ADC, the required noise performance of the analog front-end and the summing stage preceding the ADC and therefore the total power consumption and area of the chip can be further relaxed without jeopardizing the recovery performance.

### C. Comparison and Future Work

Table IV summarizes the performance of the system and presents a comparison with published works.

TABLE IV
COMPARISON OF SYSTEM PERFORMANCE WITH PUBLISHED LITERATURE

| Parameter | [8] | [37] | [47] | [48] | [49] | [50] | This Work |
|---|---|---|---|---|---|---|---|
| Technology [μm CMOS] | 0.09 | 0.13 | 0.5 | 0.18 | 0.5 | 0.5 | 0.18 |
| Power supply [V] | 0.6 | 1.2 | 3.3 | 1.8 | — | 3 | 1.2 |
| Compression method | DCS | PWL Spike det. | Spike det. | AP det. | DWT Spike det. | Spike det. | MCS |
| Number of channels | 1 | 1 | 100 | 16 | 32 | 32 | 16 |
| Compression area per channel [mm²] | 0.103 | 0.080 | <0.160 | >0.0475 | 0.18 | 0.12 | 0.008 |
| Compression power per channel [μW] | 1.9 | 1.18 | 27 | >96 | 95 | 75 | 0.95 |
| Sampling rate per channel [kS/s] | ≦20 | 90 | 15 | 30 | 25 | 20 | 4 |
| Compression ratio | ≦10 | 125 | 150 | 48 | ≦20 | 12.5 | ≦16 |

In this table, compression power and area refer to the extra power consumption and area usage of the signal digitization, compression and thresholding blocks which are commonly added to the total power consumption and area of the analog front-end. The authors in [8] apply compressive sensing on a single-channel pre-recorded EEG data by acquiring measurements in the digital domain. The power saving is significant while the area overhead is not addressed. Due to the youthfulness of the field and the lack of similar electronic architectures that use CS in brain implants, we have compared our results with a Discrete Wavelet Transform (DWT)-based design [49] for intra-cortical implants and several additional systems based on spike/AP detection [37], [47], [48], [50] for implantable neural recording applications. While the design in [49] mainly addresses the area-efficiency of the implantable system and proposes an architecture that sequentially evaluates the DWT of the multichannel data in the digital domain, our results outperform this approach in terms of area and power efficiency.

In addition, high compression ratios are achieved by means of the following thresholding and redundancy removal stages, while the DWT by itself does not result in any data compression. Thresholding, however, results in a significant loss of the signal in non-spiking regions while a more precise recovery is achieved at much lower compression ratios (e.g., at CR=2 in [49]). The chip includes several memory registers containing threshold values of different channels and additional blocks such as controllers, address generator and buffer units which degrade the power and area efficiency of the system.

Some of the reported spike detector systems achieve significant data reduction [37], [47] with negligible overhead in terms of compression power and area [37]. However, the patient-specific threshold setting in such systems can result in design complexity in a real neural interface in addition to the loss of signal in non-spiking regions. Furthermore, the transmitted signal may not be acceptable to the clinicians who usually prefer to have access to the entire iEEG data, even though somewhat lossy, for a thorough neurological examination.

As a final remark, the method presented in this invention can also be implemented in a digital framework, similar to the concept presented in [8], but in a multichannel fashion. By using an ADC per channel and applying the random controlling signals to the digital outputs of channels, which pass through a single digital accumulator block, the full array will be encoded into a digital compressed data. Consequently, any possible advantage of applying CS in the digital domain as expressed in [8] can be exploited in a more area-efficient approach, compared to applying CS per channel. However, the system implemented in this invention benefits from using a single ADC per array which results in a small effective area per channel.

As an alternative approach to the proposed method, implementing an intricate ADC which performs the randomized summation of the channels' outputs will eliminate the summing stage and improve the area efficiency of the multichannel compressive sensing system. Further comparison and characterization of the proposed approaches is required to achieve an optimal model for multichannel signal compression.

The present invention thus relates to a new multichannel architecture and method for compressive recording of biological signals. In particular, a new multichannel architecture and method for compressive recording of cortical signals at the surface of the cortex is proposed. In addition to the area efficiency, the proposed method is easily adaptable to different compression ratios, depending on the sparsity of the input signals. The power efficiency resulting from the compressive sensing methodology in addition to the minimal area cost, make this approach highly relevant for power- and area-constrained multichannel sparse signal acquisition. This approach can be investigated in other applications than neural recording, which require data recording from multiple nodes.

Extensive system-level analysis and simulations confirm the relevance and efficiency of the system for high-density recording applications, compared to alternative compression methods.

Having described now the preferred embodiments of this invention, it will be apparent to one of skill in the art that other embodiments incorporating its concept may be used. This invention should not be limited to the disclosed embodiments, but rather should be limited only by the scope of the appended claims.

### REFERENCES

[1] M. Mollazadeh, K. Murari, G. Cauwenberghs, and N. V. Thakor, “Wireless micropower instrumentation for multimodal acquisition of electrical and chemical neural activity,” IEEE Trans. Biomed. Circuits Syst., vol. 3, no. 6, pp. 388-397, 2009.
[2] M. J. Cook et al., “Prediction of seizure likelihood with a long-term, implanted seizure advisory system in patients with drug-resistant epilepsy: A first-in-man study,” Lancet Neurol., vol. 12, pp. 563-571,2013.
[3] A. B. Schwartz, X. T. Cui, D. J. Weber, and D. W. Moran, “Braincontrolled interfaces: Movement restoration with neural prosthetics,” Neuron, vol. 52, pp. 205-220, 2006.
[4] D. Donoho, “Compressed sensing,” IEEE Trans. Inf. Theory, vol. 52, no. 4, pp. 1289-1306, 2006.
[5] E. J. Candès, “Compressive sampling,” in Proc. Int. Congress of Mathematicians, 2006, pp. 1433-1452.
[6] H. Mamaghanian, N. Khaled, D. Atienza, and P. Vandergheynst, “Compressed sensing for real-time energy-efficient ECG compression on wireless body sensor nodes,” IEEE Trans. Biomed. Eng., vol. 58, no. 9, pp. 2456-2466, 2011.
[7] A. M. R. Dixon, E. G. Allstot, D. Gangopadhyay, and D. J. Allstot, “Compressed sensing system considerations for ECG and EMG wireless biosensors,” IEEE Trans. Biomed. Circuits Syst., vol. 6, no. 2, pp. 156-166, 2012.
[8] F. Chen, A. P. Chandrakasan, and V. Stojanovic, “Design and analysis of a hardware-efficient compressed sensing architecture for data compression in wireless sensors,” IEEE J. Solid-State Circuits, vol. 47, pp. 744-756, 2012.
[9] M. H. Kamal, M. Shoaran, Y. Leblebici, A. Schmid, and P. Vandergheynst, “Compressive multichannel cortical signal recording,” in Proc. IEEE Int. Conf. Acoustics, Speech and Signal Processing, 2013, pp. 4305-4309.
[10] G. A. Worrell et al., “High-frequency oscillations in human temporal lobe: Simultaneous microwire and clinical macroelectrode recordings,” Brain, vol. 131, pp. 928-937, 2008.
[11] J. J. V. Gompel et al., “Phase I trial: Safety and feasibility of intracranial electroencephalography using hybrid subdural electrodes containing macro- and microelectrode arrays,” Neurosurg. Focus, vol. 25, 2008.
[12] M. Stead et al., “Microseizures and the spatiotemporal scales of human partial epilepsy,” Brain, vol. 133, pp. 2789-2797, 2010.
[13] M. S. Fifer, S. Acharya, H. L. Benz, M. Mollazadeh, N. E. Crone, and N. V. Thakor, “Toward electrocorticographic control of a dexterous upper limb prosthesis: Building brain-machine interfaces,” IEEE Pulse, vol. 3, pp. 38-42, 2012.
[14] X. Chen, Z. Yu, S. Hoyos, B. M. Sadler, and J. Silva-Martinez, “A sub-Nyquist rate sampling receiver exploiting compressive sensing,” IEEE Trans. Circuits Syst. I, Reg. Papers, vol. 58, no. 3, pp. 507-520, 2010.
[15] J. N. Laska, S. Kirolos, M. F. Duarte, T. S. Ragheb, R. G. Baraniuk, and Y. Massoud, “Theory and implementation of an analog-to-information converter using random demodulation,” in Proc. IEEE Int. Symp. Circuits and Systems, 2007, pp. 1959-1962.
[16] M. Shoaran, C. Pollo, Y. Leblebici, and A. Schmid, “Design techniques and analysis of high-resolution neural recording systems targeting epilepsy focus localization,” in Proc. Int. Conf. IEEE Engineering in Medicine and Biology Soc., 2012, pp. 5150-5153.
[17] R. G. Baraniuk, “Compressive sensing,” IEEE Signal Process. Mag., vol. 24, no. 4, pp. 118-121, 2007.
[18] E. J. Candès and J. Romberg, “Sparsity and incoherence in compressive sampling,” Inverse Problems, vol. 23, no. 3, pp. 969-985, 2007.
[19] E. J. Candès and T. Tao, “Decoding by linear programming,” IEEE Trans. Inf. Theory, vol. 51, no. 12, pp. 4203-4215, 2005.
[20] S. Qian and D. Chen, “Discrete Gabor transform,” IEEE Trans. Signal Process., vol. 41, no. 7, pp. 2429-2438, 1993.
[21] M. Kowalski and B. Torrésani, “Sparsity and persistence: Mixed norms provide simple signal models with dependent coefficients,” Signal, Image, Video Process., vol. 3, no. 3, pp. 251-264, 2009.
[22] H. Rezaee-Dehsorkh, N. Ravanshad, R. Lofti, K. Mafinezhad, and A. M. Sodagar, “Analysis and design of tunable amplifiers for implantable neural recording applications,” IEEE J. Emerg. Select. Topics Circuits Syst., vol. 1, no. 4, pp. 546-556, 2011.
[23] K. A. Ng and Y. P. Xu, “A compact, low input capacitance neural recording amplifier,” IEEE Trans. Biomed. Circuits Syst., vol. 7, no. 5, pp. 610-620, 2013.
[24] R. R. Harrison and C. Charles, “A low-power low-noise CMOS amplifier for neural recording applications,” IEEE J. Solid-State Circuits, vol. 38, no. 6, pp. 958-965, 2003.
[25] S. Amico, M. Conta, and A. Baschirotto, “A 4.1-mW 10-MHz fourthorder source follower-based continuous-time filter with 79-dB DR,” IEEE J. Solid-State Circuits, vol. 41, no. 12, pp. 2713-2719, 2006.
[26] K. S. Guillory and R. A. Normann, “A 100-channel system for real time detection and storage of extracellular spike waveforms,” J. Neurosci. Methods, vol. 91, pp. 21-29, 1999.
[27] M. S. Chae, W. Liu, and M. Sivaprakasam, “Design optimization for integrated neural recording systems,” IEEE J. Solid-State Circuits, vol. 43, no. 3, pp. 1931-1939, 2008.
[28] S. Rabii and B. A. Wooley, “A 1.8-V digital-audio sigma-delta modulator in 0.8-m CMOS,” IEEE J. Solid-State Circuits, vol. 32, no. 6, pp. 783-796, 1997.
[29] M. Shoaran, M. M. Lopez, V. S. R. Pasupureddi, Y. Leblebici, and A. Schmid, “A low-power area-efficient compressive sensing approach for multi-channel neural recording,” in Proc. IEEE Int. Symp. Circuits and Systems, 2013, pp. 2191-2194.
[30] R. Schreier, J. Silva, J. Steensgaard, and G. C. Temes, “Design-oriented estimation of thermal noise in switched-capacitor circuits,” IEEE Trans. Circuits Syst. I, Reg. Papers, vol. 52, no. 11, pp. 2358-2368, 2005.
[31] W. Wattanapanitch, M. Fee, and R. Sarpeshkar, “An energy-efficient micropower neural recording amplifier,” IEEE Trans. Biomed. Circuits Syst., vol. 1, no. 2, pp. 136-147, 2007.
[32] M. Steyaert, W. Sansen, and C. Zhongyuan, “A micropower low-noise monolithic instrumentation amplifier for medical purposes,” IEEE J. Solid-State Circuits, vol. 22, no. 6, pp. 1163-1168, 1987.
[33] T. M. Seese, H. Harasaki, G. M. Saidel, and C. Davies, “Characterization of tissue morphology, angiogenesis, and temperature in the adaptive response of muscle tissue in chronic heating,” Lab. Invest., vol. 78, pp. 1553-1562, 1998.
[34] G. Higgins, S. Faul, R. P. McEvoy, B. McGinley, M. Glavin, W. P. Marnane, and E. Jones, “EEG compression using JPEG2000: How much loss is too much?,” in Proc. Int. Conf. IEEE Engineering in Medicine and Biology Soc., 2010, pp. 614-617.
[35] A. Bragin, I. Mody, C. L. Wilson, and J. Engel, “Local generation of fast ripples in epileptic brain,” J. Neurosci., pp. 2012-2021, 2002.
[36] J. D. Jirsch, E. Urrestarazu, P. LeVan, A. Olivier, F. Dubeau, and J. Gotman, “High-frequency oscillations during human focal seizures,” Brain, vol. 129, pp. 1593-1608, 2006.
[37] A. Rodriguez-Perez, J. Ruiz-Amaya, M. Delgado-Restituto, and A. Rodriguez-Vazquez, “A low-power programmable neural spike detection channel with embedded calibration and data compression,” IEEE Trans. Biomed. Circuits Syst., vol. 6, no. 2, pp. 87-100, 2012.
[38] F. Shahrokhi, K. Abdelhalim, D. Serletis, P. L. Carlen, and R. Genov, “The 128-channel fully differential digital integrated neural recording and stimulation interface,” IEEE Trans. Biomed. Circuits Syst., vol. 4, no. 3, pp. 149-161, 2010.
[39] S. Rai, J. Holleman, J. N. Pandey, F. Zhang, and B. Otis, “A 500 W neural tag with 2 V AFE and frequency-multiplying MICS/ISM FSK transmitter,” in Proc. IEEE Int. Solid-State Circuits Conf., Dig. Tech. Papers, 2009, pp. 212-213, 213a.
[40] V. Majidzadeh, A. Schmid, and Y. Leblebici, “Energy efficient lowloise neural recording amplifier with enhanced noise efficiency factor,” IEEE Trans. Biomed. Circuits Syst., vol. 5, no. 3, pp. 262-271,2011.
[41] J. Aziz, K. Abdelhalim, R. Shulyzki, R. Genov, B. Bardakjian, M. Derchansky, D. Serletis, and P. Carlen, “256-channel neural recording and delta compression microsystem with 3D electrodes,” IEEE J. Solid-State Circuits, vol. 44, no. 3, pp. 995-1005, 2009.
[42] L. Wen-Sin, Z. Xiaodan, Y. Libin, and L. Yong, “A 1-v 60-uw 16-channel interface chip for implantable neural recording,” in Proc. IEEE Custom Integrated Circuits Conf., 2009, pp. 507-510.
[43] K. Abdelhalim, H. Jafari, L. Kokarovtseva, J. L. P. Velazquez, and R. Genov, “64-channel UWB wireless neural vector analyzer SoC with a closed-loop phase synchrony-triggered neurostimulator,” IEEE J. Solid-State Circuits, vol. 48, no. 10, pp. 2494-2510, 2013.
[44] K. Abdelhalim, L. Kokarovtseva, J. L. Perez Velazquez, and R. Genov, “915-MHz FSK/OOK wireless neural recording SoC with 64 mixedsignal FIR filters,” IEEE J. Solid-State Circuits, vol. 48, no. 10, pp. 2478-2493, 2013.
[45] F. Chen, F. Lim, O. Abari, A. Chandrakasan, and V. Stojanovic, “Energy-aware design of compressed sensing systems for wireless sensors under performance and reliability constraints,” IEEE Trans. Circuits Syst. I, Reg. Papers, vol. 60, no. 3, pp. 650-661, 2013.
[46] V. K. Goyal, A. K. Fletcher, S. Rangan, A. Chandrakasan, and V. Stojanovic, “Compressive sampling and lossy compression,” IEEE Signal Process. Mag., vol. 25, no. 2, pp. 48-56, 2008.
[47] R. R. Harrison, P. T. Watkins, R. J. Kier, R. O. Lovejoy, D. J. Black, B. Greger, and F. Solzbacher, “A low-power integrated circuit for a wireless 100-electrode neural recording system,” IEEE J. Solid-State Circuits, vol. 42, no. 1, pp. 123-133, 2007.
[48] B. Gosselin, A. E. Ayoub, J. F. Roy, M. Sawan, F. Lepore, A. Chaudhuri, and D. Guitton, “A mixed-signal multichip neural recording interface with bandwidth reduction,” IEEE Trans. Circuits Syst. I, Reg. Papers, vol. 3, no. 3, pp. 129-141,2009.
[49] A. M. Kamboh, K. G. Oweiss, and A. J. Mason, “Resource constrained VLSI architecture for implantable neural data compression systems,” in Proc. IEEE Int. Symp. Circuits and Systems, 2009, pp. 1481-1484.
[50] R. H. Olsson and K. D. Wise, “A three-dimensional neural recording microsystem with implantable data compression circuitry,” IEEE J. Solid-State Circuits, vol. 40, no. 12, pp. 2796-2804, 2005.

1. Method for compressing a plurality of biological signals provided by a plurality of measurement devices through a plurality of measuring channels, the method comprising the steps of:
forming a multichannel signal matrix XεR^{d×N} from the plurality of biological signals where d is a dimension of the biological signal in each channel and in a time-window and N is the number of channels, R meaning a set of real numbers;
obtaining a multichannel measurement vector Y by acquiring M=p/d measurements from each column of the multichannel signal matrix X, to acquire M measurements at each time-window from all channels, where p<<d×N.

2. Method according to claim 1, further including the step of:
applying a reshaping operator [symbol] :R^{d×N}→R^{d·N} to the multichannel signal matrix X to transpose said matrix to form a vector of concatenated columns of the multichannel signal matrix X.

3. Method according to claim 2, wherein the multichannel measurement vector Y is obtained by applying a measurement matrix φ_{MC} to the vector of concatenated columns of the multichannel signal matrix X.

4. Method according to claim 3, wherein the measurement matrix φ_{MC} is represented in matrix form as follows:
$${Φ}_{MC} = [ \begin{matrix}{Φ}_{1} & 0 ⋯ & 0 \\ ⋮ & ⋱ & ⋮ \\ 0 & ⋯ & {Φ}_{d}\end{matrix} ] , {Φ}_{i} = [ \begin{matrix}{ϕ}_{i}^{1 , 1} & ⋯ & {ϕ}_{i}^{1 , N} \\ ⋮ & ⋱ & ⋮ \\ {ϕ}_{i}^{M , 1} & ⋯ & {ϕ}_{i}^{M , N}\end{matrix} ] ,$$
where φᵢεR^{M×N} and φᵢ^{k,l} is uniformly selected from {0,1}.

5. Method according to claim 4, wherein the multichannel measurement vector Y is determined as follows:
Y=φ_{MC} [symbol] (X)

6. Method according to claim 1, further including the step of:
recovering a multichannel signal \hat{X} from the multichannel measurement vector Y using an l_{1,2} mixed norm as follows:
$$\hat{X} = {Ψ}_{MC} {argmin}_{{α}_{MC} ∈ {R}^{d \cdot N}} \vert \vert {α}_{MC} {\vert \vert}_{1 , 2} s . t . Y = {Φ}_{MC} 𝒫 ( {Ψ}_{MC} {α}_{MC} )$$
where argmin is argument of the minimum, ∥•∥_{1,2} models group sparsity according to the l_{1,2} mixed norm, α_{MC} is a Gabor transform coefficients, [symbol] is the reshaping operator and ψ_{MC} is a sparsity matrix.

7. Method according to claim 1, further including the step of:
recovering a multichannel neural signal \hat{X} from the multichannel measurement vector Y using an l₁ norm as follows:
$$\hat{X} = {argmin}_{X ∈ {R}^{d \times N}} \vert \vert {Ψ}_{MC}^{T} {X}_{vec} {\vert \vert}_{1} s . t . Y = {Φ}_{MC} 𝒫 ( X )$$
where X_{vec} is the vector form of X which includes the concatenation of its columns, and the l₁ norm of a vector ξ is defined as ∥ξ∥₁=Σᵢ|ξᵢ|.

8. Implantable sensor including circuitry configured to carry out the method according to claim 1.

9. Implantable system including an implantable transmitter and at least one implantable sensor according to claim 8, the at least one implantable sensor being connected to the implantable transmitter to communicate the compressed signal to the implantable transmitter for transmission to an external receiver.

10. System for compressing a plurality of biological signals and recovering biological signals, the system including the implantable system according to claim 9 and a receiver for receiving a transmitted compressed signal.

11. System according to claim 10, further including a processor configured to receive the compressed signal from the receiver and to process the compressed signal to reconstruct the captured biological signals.

12. Implantable sensor including:
a plurality of electrodes for capturing a biological signal,
a plurality of amplifiers, each amplifier being connected to a single electrode to amplify the signal captured by each electrode,
a random summing circuit for receiving each of the amplified signals, for randomly selecting samples of the amplified captured signals and summing said samples to provide a multichannel measurement signal, and
a single analog-to-digital converter for receiving and digitizing the multichannel measurement signal to provide a digital compressed signal.

13. Implantable sensor according to claim 12, wherein each amplifier is a low-noise amplifier including a band-pass transfer function.

14. Implantable sensor according to claim 12, further including a plurality of low-pass filters to limit a high cut-off frequency of the amplified captured signal, each low-pass filter being connected to a single amplifier to receive the amplified captured signal.

15. Implantable sensor according to claim 14, further including a plurality of additional amplifiers, each additional amplifier being connected to a single low-pass filter to amplify the signal provided by said low-pass filter.

16. Implantable sensor according to claim 15, further including a plurality of buffered sample-and-hold circuits, each buffered sample-and-hold circuit being connected to a single additional amplifier to receive the signal amplified by said additional amplifier.

17. Implantable system including an implantable transmitter and at least one implantable sensor according to claim 12, the least one implantable sensor being connected to the implantable transmitter to communicate the compressed signal to the implantable transmitter for transmission to an external receiver.

18. System for compressing a plurality of biological signals and recovering biological signals, the system including the implantable system according to claim 16 and a receiver for receiving a transmitted compressed signal.

19. System according to claim 18, further including a processor configured to receive the compressed signal from the receiver and to process the compressed signal to reconstruct the captured biological signals.
